(19) United States
(12) Patent Application Publication
(10) Pub. No.: US 2018/0214547 A1
(43) Pub. Date: Aug. 2, 2018

(54) TREATMENT OF CANCER USING A COMBINATION OF AN ANTI-PD-1 ANTIBODY AND IL-6

(71) Applicant: BRISTOL-MYERS SQUIBB COMPANY, Princeton, NJ (US)

(72) Inventor: Katherine E. Lewis, Lake Forest Park, WA (US)

(21) Appl. No.: 15/743,013

(22) PCT Filed: Jul. 8, 2016
(86) PCT No.: PCT/US2016/041434

§ 371 Date: Jan. 9, 2018

Related U.S. Application Data
(60) Provisional application No. 62/190,322, filed on Jul. 9, 2015.

(51) Int. Cl. A61K 39/395; A61K 38/20; C07K 16/28; A61P 35/00

(52) U.S. Cl. CPC A61K 39/39558; A61K 38/204; A61K 2039/545; A61P 35/00; A61K 2300/00; C07K 16/2818

(57) ABSTRACT

This disclosure provides a method for treating a subject afflicted with a cancer, which method comprises administering to the subject therapeutically effective amounts of: (a) an anti-cancer agent which is an antibody or an antigen-binding portion thereof that specifically binds to a Programmed Death-1 (PD-1) receptor and inhibits PD-1 activity; and, (b) IL-6.

Throughout this application, various publications are referenced in parentheses by author name and date, or by patent No. or patent Publication No. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

This invention relates to methods for treating cancer in a subject comprising administering to the subject a combination of an anti-cancer agent which is an anti-Programmed Death-1 (PD-1) or anti-Programmed Death Ligand-1 (PD-L1) antibody and IL-6.

### BACKGROUND OF THE INVENTION

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all of these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody (Ab), ipilimumab (YERVOY®), that binds to and inhibits CTLA-4 for the treatment of patients with advanced melanoma (Hodi et al., 2010) and the development of Abs such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of Ab inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-L1 and PD-L2), thereby blocking the downregulation of antitumor T-cell functions (U.S. Pat. No. 8,008,449; Wang et al., 2014). Nivolumab has shown activity in a variety of advanced solid tumors including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

Interleukin (IL)-6 is a 26 kDa protein that is produced by T-lymphocytes, monocytes, fibroblasts, endothelial cells and keratinocytes (Le and Vilcek, 1989; reviewed in Gilbert et al, 2012). IL-6 is a pleiotropic cytokine with stimulatory actions on hematopoiesis via its role in the production of mature myeloid and megakaryocytic cells, as well as its stimulatory actions on the immune system and responses to infections and inflammation via its role in the induction of acute phase proteins (Le and Vilcek, 1989). IL-6 exerts its biological effects via a signaling cascade that is tightly regulated (Rose-John S, 2012; Silver J S and Hunter C A, 2010). Classical IL-6 signaling occurs when IL-6 binds to the membrane-spanning, non-signaling IL-6 receptor (IL-6R) subunit. The role of IL-6 signaling in inflammation is complex. Although IL-6 has anti-inflammatory activities in some settings, its role in immunity and promoting inflammation are clear. It is the latter properties that can be utilized to treat cancer. In this respect, IL-6 has been shown to have numerous activities which promote antitumor immunity, i.e. enhance CD8+ effector T cell trafficking, enhance cytotoxic T cell responses, enhance NK cell responses, stimulate the generation of mature B cells, provide important survival and proliferative signals to various leukocyte populations and suppress regulatory T cells (reviewed in Gilbert et al, 2012).

Consistent with these activities and findings, there is a large body of data to support the use of IL-6 as an antitumor agent. Numerous reports have shown that tumor cells transfected with the IL-6 gene have reduced tumorigenicity, enhanced immunogenicity and decreased metastatic potential in a number of different in vivo tumor models (Mullen C A et al, 1992; Progador A et al, 1992; Sun W H et al, 1992; Cao et al, 1995; Bhanumathy K K et al, 2014). In addition, the use of recombinant (r)IL-6 delivery has also been demonstrated to have antitumor activities across numerous tumor types in preclinical models (Givon et al, 1992; Mulé et al, 1990; Eisenthal et al, 1993; Marcus et al, 1995). These data have demonstrated that IL-6 delivery, whether via gene transfection or via treatment with recombinant IL-6 (rIL-6), can result in growth inhibition of subcutaneous tumors, reduction in the number of metastases and/or prolong survival in preclinical models of cancer. In addition, it has recently been reported that exercise-induced IL-6 plays a key role in the antitumor activity observed with exercise (Pedersen et al, 2016). Exercise is known to elevate levels of IL-6 and Pedersen et al scientists have determined that it is this increase in IL-6 that is in part responsible for the antitumor activity and chemoprotection observed. Several mechanisms that could explain the antitumor role of rIL-6 have been demonstrated in in vitro and in vivo systems, including direct effects on tumor cells and indirect effects on host defenses, such as its role as a co-activator of T and B lymphocytes, its ability to enhance T cell infiltration into the tumor, its ability to induce NK cell responses and cytotoxic T cell activities, and its role in the suppression of Treg activity and/or expression (Cao et al, 1995; Kishimoto T, 1989; Marcus et al, 1995; Eisenthal et al, 1993; Lin et al, 2012; Bhanumathy K K et al, 2014). Given the numerous preclinical studies and mechanistic studies demonstrating IL-6-mediated tumor cell destruction and growth inhibition, human clinical trials were conducted to evaluate the value of this cytokine in the immunotherapy of cancer, in the form of recombinant human (rh)IL-6 (Veldhuis et al, 1996). Preliminary data from trials investigating rhIL-6 in renal cell carcinoma and melanoma showed low response rates (8-14%) indicating that there may actually be limited potential for rhIL-6 as a cancer therapeutic (Veldhuis et al, 1996).

However, in combination with other agents, the antitumor potential of IL-6 could be enhanced. In fact, the combination of IL-6 with other cytokines and/or B7-1 costimulation in vitro has demonstrated significant combinatorial advantages in inducing the expansion of effector lymphocytes, the generation of specific lytic activities and the induction of IFN-gamma, all associated with an effective antitumor response (Gajewski et al, 1995; Qu et al, 1999).

In addition to its role as an antitumor therapeutic, IL-6 delivery is able to reduce deleterious side effects resulting from chemotherapy used during cancer therapy, such as chemotherapy-induced bone marrow depression and thrombocytopenia (Veldhuis et al, 1996) and chemotherapy-induced neuropathy (Callizot et al, 2008). Thus, the use of IL-6 in the treatment of cancer, i.e. as an antitumor agent, as a therapeutic to protect against potential cancer treatment-related side effects as well as an agent to increase and/or enhance the immune response, would be anticipated to be of benefit to the cancer patient.

Thus, the present invention relates to the use of soluble human IL-6 (hIL-6) in combination with a PD-1/PD-L1 antagonist for the treatment of cancer.

### SUMMARY OF THE INVENTION

The present disclosure provides a method for treating a subject afflicted with a cancer comprising administering to the subject a therapeutically effective amounts of: (a) an antibody or an antigen-binding portion thereof that specifically binds to and inhibits the PD-1/PD-L1 signaling pathway; and, (b) IL-6. In some embodiments, the anti-cancer agent is an antibody or an antigen-binding portion thereof that binds specifically to a PD-1 receptor and inhibits PD-1 activity and is administered by infusion for less than 60 minutes (e.g., about 30 minutes). In some embodiments, the other IL-6 is administered by infusion for less than 90 minutes (e.g., about 60 or about 30 minutes). In certain preferred embodiments of any of the therapeutic methods disclosed herein, the anti-PD-1 Ab is nivolumab. In other embodiments, the anti-PD-1 Ab is pembrolizumab.

### BRIEF DESCRIPTION OF THE DRAWINGS

FIG. 1 shows the combination efficacy observed in this well-established pre-clinical mouse tumor model suggesting that co-treatment with PD-1 mAb and recombinant IL-6 could be an effective method for the treatment of cancer.

FIG. 2 shows individual mouse tumor volume data showing antitumor activity of mPD-1 mAb and hIL-6, alone or in combination, in the CT-26 tumor model.

FIG. 3 shows the area-under-the-curve (AUC) values for tumor volumes through Day 24 showing antitumor activity of mPD-1 mAb and hIL-6, alone or in combination, in the CT-26 tumor model.

FIG. 4 shows survival proportions for mice treated with mIgG1, or mPD-1 mAb or hIL-6 alone and in combination in the CT-26 tumor model.

FIG. 5 shows the mean and median tumor volumes by group showing antitumor activity of mPD-1 mAb and hIL-6, alone or in combination, in the MC-38 tumor model.

FIG. 6 shows individual mouse tumor volume data showing antitumor activity of mPD-1 mAb and hIL-6, alone or in combination, in the MC-38 tumor model.

FIG. 7 shows the area-under-the-curve (AUC) values for tumor volumes through Day 27 showing antitumor activity of mPD-1 mAb and hIL-6, alone or in combination, in the MC-38 tumor model.

FIG. 8 shows the survival proportions for mice treated with mIgG1, or mPD-1 mAb or hIL-6 alone and in combination in the MC-38 tumor model.

FIG. 9 shows the mean and median tumor volumes by group showing antitumor activity of mPD-1 mAb and mIL-6, alone or in combination, in the CT-26 tumor model.

FIG. 10 shows the individual mouse tumor volume data showing antitumor activity of mPD-1 mAb and mIL-6, alone or in combination, in the CT-26 tumor model.

FIG. 11 shows the area-under-the-curve (AUC) values for tumor volumes through Day 21 showing antitumor activity of mPD-1 mAb and mIL-6, alone or in combination, in the CT-26 tumor model.

FIG. 12 shows the survival proportions for mice treated with mIgG1, or mPD-1 mAb or mIL-6 alone and in combination in the CT-26 tumor model.

FIG. 13 shows the mean and median tumor volumes by group showing antitumor activity of mPD-1 mAb and mIL-6, alone or in combination, in the CT-26 tumor model.

FIG. 14 shows the individual mouse tumor volume data showing antitumor activity of mPD-1 mAb and mIL-6, alone or in combination, in the CT-26 tumor model.

FIG. 15 shows the area-under-the-curve (AUC) values for tumor volumes through Day 23 and Day 27 showing antitumor activity of mPD-1 mAb and mIL-6, alone or in combination, in the CT-26 tumor model.

FIG. 16 shows the percentage of Foxp3+ regulatory T cells (Treg) as a proportion of live CD45+ cells in tumors (at Day 15) from mice treated with mIgG1, or mPD-1 mAb or mIL-6 alone and in combination in the CT-26 tumor model.

FIG. 17 shows the mean and median tumor volumes by group showing antitumor activity of mPD-1 mAb and mIL-6 (3 or 30 μg/kg), alone or in combination, in the CT-26 tumor model.

FIG. 18 shows the individual mouse tumor volume data showing antitumor activity of mPD-1 mAb and mIL-6 (3 or 30 μg/kg), alone or in combination, in the CT-26 tumor model.

FIG. 19 shows the area-under-the-curve (AUC) values for tumor volumes through Day 19 and Day 22 showing antitumor activity of mPD-1 mAb and mIL-6 (3 or 30 μg/kg), alone or in combination, in the CT-26 tumor model.

FIG. 20 shows the concentration of interferon gamma in tumor supernatants at Day 15 from CT-26 bearing mice, expressed as pg/mL per gram tumor weight.

### DETAILED DESCRIPTION OF THE INVENTION

The present invention relates to methods for treating a cancer patient comprising administering to the patient a combination of an anti-PD-1 antibody and IL-6. The invention is based on the findings that the co-administration of human or mouse IL-6 and an anti-PD-1 monoclonal antibody (mAb) in reliable animal models of cancer is efficacious in its ability to reduce tumor growth and progression, promote tumor regression and increase survival as compared to isotype control-treated groups, or groups treated with IL-6 or a PD-1 mAb alone. Because IL-6 is a potent pro-inflammatory cytokine it is critical to be able to use an efficacious dose of IL-6 that has been found to be safe and to not result in adverse events that would otherwise limit its use.

### Terms

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 may, however, have cross-reactivity to other antigens, such as PD-1 molecules from different species. Moreover, an isolated Ab may be substantially free of other cellular material and/or chemicals.

The term “monoclonal antibody” (“mAb”) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. MAbs may be produced by hybridoma, recombinant, transgenic or other techniques known to those skilled in the art.

A “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human” Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “antigen-binding portion” of an Ab (also called an “antigen-binding fragment”) refers to one or more fragments of an Ab that retain the ability to bind specifically to the antigen bound by the whole Ab.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

The term “interleukin-6” or “IL-6” (also known as interferon-β₂; B-cell differentiation factor; B-cell stimulatory factor-2; hepatocyte stimulatory factor; hybridoma growth factor; and plasmacytoma growth factor) is a multifunctional cytokine involved in numerous biological processes such as the regulation of the acute inflammatory response, the modulation of specific immune responses including B- and T-cell differentiation, bone metabolism, thrombopoiesis, epidermal proliferation, menses, neuronal cell differentiation, neuroprotection, aging, cancer, and the inflammatory reaction occurring in Alzheimer's disease. See A. Papassotiropoulos et al, Neurobiology of Aging, 22:863-871 (2001). IL-6 is a member of a family of cytokines that promote cellular responses through a receptor complex consisting of at least one subunit of the signal-transducing glycoprotein gp130 and the IL-6 receptor (“IL-6R”) (also known as gp80). The IL-6R may also be present in a soluble form (“sIL-6R”). IL-6 binds to IL-6R, which then dimerizes the signal-transducing receptor gp130. See Jones, S A, J. Immunology, 175:3463-3468 (2005). In humans, the gene encoding IL-6 is organized in five exons and four introns, and maps to the short arm of chromosome 7 at 7p21. Translation of IL-6 RNA and post-translational processing result in the formation of a 21 to 28 kDa protein with 184 amino acids in its mature form. See A. Papassotiropoulos, et al, Neurobiology of Aging, 22:863-871 (2001). The term IL-6 includes all forms of IL-6 including but not limited to a mutein, isoform, fused protein, functional derivative, active fraction or circularly permutated derivative or a salt thereof.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Anti-PD-1 and Anti-PD-L1 Antibodies

The methods of the invention include treatment of a cancer with an inhibitor of PD-1 or PD-L1. In one aspect, the first antibody is an anti-PD1 antibody or an anti-PD-L1 antibody. PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

HuMAbs that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a KD of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 Abs useful for the present invention include mAbs that bind specifically to human PD-1 and exhibit at least one, preferably at least five, of the preceding characteristics.

In one embodiment, the anti-PD-1 Ab is nivolumab. Nivolumab (also known as “Opdivo®”; formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-L1 and PD-L2), thereby blocking the downregulation of antitumor T-cell functions (U.S. Pat. No. 8,008,449; Wang et al., 2014 Cancer Immunol Res. 2(9):846-56).

In another embodiment, the anti-PD-1 Ab is pembrolizumab. Pembrolizumab (also known as “Keytruda®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also http://www.cancer.gov/drugdictionary?cdrid=695789 (last accessed: Dec. 14, 2014). Pembrolizumab has been approved by the FDA for the treatment of relapsed or refractory melanoma.

In other embodiments, the anti-PD-1 Ab is MEDI0608 (formerly AMP-514), which is a monoclonal antibody. MEDI0608 is described, for example, in U.S. Pat. No. 8,609,089B2 or in http://www.cancer.gov/drugdictionary?cdrid=756047 (last accessed Dec. 14, 2014).

In some embodiments, the anti-PD-1 antibody is Pidilizumab (CT-011), which is a humanized monoclonal antibody. Pidilizumab is described in U.S. Pat. No. 8,686,119 B2 or WO 2013/014668 A1.

In other embodiments, the anti-PD-1 antibody is BGB-A317 which is a humanized monoclonal antibody. BGB-A317 is described in U.S. Pat. No. 8,735,553.

Anti-PD-1 Abs useful for the disclosed compositions also include isolated Abs that bind specifically to human PD-1 and cross-compete for binding to human PD-1 with nivolumab (see, e.g., U.S. Pat. Nos. 8,008,449 and 8,779,105; WO 2013/173223). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar to those of nivolumab by virtue of their binding to the same epitope region of PD-1. Cross-competing Abs can be readily identified based on their ability to cross-compete with nivolumab in standard PD-1 binding assays such as Biacore analysis, ELISA assays or flow cytometry (see, e.g., WO 2013/173223).

In certain embodiments, the Abs that cross-compete for binding to human PD-1 with, or bind to the same epitope region of human PD-1 as, nivolumab are mAbs. For administration to human subjects, these cross-competing Abs can be chimeric Abs, or humanized or human Abs. Such chimeric, humanized or human mAbs can be prepared and isolated by methods well known in the art.

Anti-PD-1 Abs useful for the compositions of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab.

Anti-PD-1 Abs suitable for use in the disclosed compositions are Abs that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole Abs in inhibiting ligand binding and upregulating the immune system. In certain embodiments, the anti-PD-1 Ab or antigen-binding portion thereof cross-competes with nivolumab for binding to human PD-1. In other embodiments, the anti-PD-1 Ab or antigen-binding portion thereof is a chimeric, humanized or human monoclonal Ab or a portion thereof. In certain embodiments, the Ab is a humanized Ab. In other embodiments, the Ab is a human Ab. Abs of an IgG1, IgG2, IgG3 or IgG4 isotype can be used.

In certain embodiments, the anti-PD-1 Ab or antigen-binding portion thereof comprises a heavy chain constant region which is of a human IgG1 or IgG4 isotype. In certain other embodiments, the sequence of the IgG4 heavy chain constant region of the anti-PD-1 Ab or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the Ab comprises a light chain constant region which is a human kappa or lambda constant region. In other embodiments, the anti-PD-1 Ab or antigen-binding portion thereof is a mAb or an antigen-binding portion thereof. In certain embodiments of any of the therapeutic methods described herein comprising administration of an anti-PD-1 Ab, the anti-PD-1 Ab is nivolumab. In other embodiments, the anti-PD-1 Ab is pembrolizumab. In other embodiments, the anti-PD-1 Ab is chosen from the human antibodies 17D8, 2D3, 4H1, 4A11, 7D3 and 5F4 described in U.S. Pat. No. 8,008,449. In still other embodiments, the anti-PD-1 Ab is MEDI0608 (formerly AMP-514), AMP-224, or Pidilizumab (CT-011).

In certain embodiments, the first antibody for the disclosed composition is an anti-PD-L1 antibody. Because anti-PD-1 and anti-PD-L1 target the same signaling pathway and have been shown in clinical trials to exhibit similar levels of efficacy in a variety of cancers, an anti-PD-L1 Ab can be substituted for the anti-PD-1 Ab in any of the therapeutic methods or compositions disclosed herein. In certain embodiments, the anti-PD-L1 Ab is BMS-936559 (formerly 12A4 or MDX-1105) (see, e.g., U.S. Pat. No. 7,943,743; WO 2013/173223). In other embodiments, the anti-PD-L1 Ab is MPDL3280A (also known as RG7446) (see, e.g., Herbst; U.S. Pat. No. 8,217,149) or MEDI4736 (Khleif, 2013).

### IL-6

The methods of the invention include treatment of a cancer with soluble IL-6. In one embodiment, the IL-6 is human IL-6. The complete human IL-6 sequence can be found under GenBank Accession No. BC015511.1. The cDNA sequence and amino acid sequence of human IL-6 are identified below as SEQ ID NO:1 and 2, respectively. In one embodiment of the present invention, the human IL-6 is produced recombinantly.

(SEQ ID NO: 1)
cccgctctggccccaccctcaccctccaacaaagatttatcaaatgtgg
gattttcccatgagtctcaatattagagtctcaacccccaataaatata
ggactggagatgtctgaggctcattctgccctcgagcccaccgggaacg
aaagagaagctctatctcccctccaggagcccagctatgaactccttct
ccacaagtaagtgcaggaaatccttagccctggaactgccagcggcggt
cgagccctgtgtgagggaggggtgtgtggcccagggagggctggcgggc
ggccagcagcagaggcaggctcccagctgtgctgtcagctcacccctgc
gctcgctcccctccggcacaggcgccttcggtccagttgccttctccct
ggggctgctcctggtgttgcctgctgccttccctgccccagtaccccca
ggagaagattccaaagatgtagccgccccacacagacagccactcacct
cttcagaacgaattgacaaacaaattcggtacatcctcgacggcatctc
agccctgagaaaggagacatgtaacaagagtaacatgtgtgaaagcagc
aaagaggcactggcagaaaacaacctgaaccttccaaagatggctgaaa
aagatggatgcttccaatctggattcaatgaggagacttgcctggtgaa
aatcatcactggtcttttggagtttgaggtatacctagagtacctccag
aacagatttgagagtagtgaggaacaagccagagctgtgcagatgagta
caaaagtcctgatccagttcctgcagaaaaaggtgggtgtgtcctcatt
ccctcaacttggtgtgggggaagacaggctcaaagacagtgtcctggac
aactcagggatgcaatgccacttccaaaagagaaggctacacgtaaaca
aaagagtctgagaaatagtttctgattgttattgttaaatctattagtt
tgtttggttggttggctctcttctgcaaaggacatcaataactgtattt
taaactatatattaactgaggtggattttaacatcaatttttaatagtg
caagagatttaaaaccaaaggcgggggggcgggcagaaaaaagtgcatc
caactccagccagtgatccacagaaacaaagaccaaggagcacaaaatg
attttaagattttagtcattgccaagtgacattcttctcactgtggttg
tttcaattctttttcctaccttttaccagagagttagttcagagaaatg
gtcagagactcaagggtggaaagaggtaccaaaggctttggccaccagt
agctggctattcagacagcagggagtagacttgctggctagcatgtgga
ggagccaaagctcaataagaaggggcctagaatgaaacccttggtgctg
atcctgcctctgccatttctacttaagccagggtttctcatatgttaac
atgcatgggaattccctgggcatcttcttgtggtgtggagtctgactta
gcaagcctcgggtgggtttgagggtcaaatttctaccaggcttatatcc
ctggtgatgctgcagaattccaggaccacacttggaggtttaaggcctt
ccacaagttacttatcccatatggtgggtctatggaaaggtgtttccca
gtcctctttacaccaccggatcagtggtctttcaacagatcctaaaggg
atggtgagagggaaactggagaaaagtatcagatttagaggccactgaa
gaacccatattaaaatgcctttaagtatgggctcttcattcatatacta
aatatgaactatgtgccaggcattatttcatatgacagaatacaaacaa
ataagatagtgatgctggtcaggcttggtggctcatgcctgtattccct
aaactttgggagcctaaggtgagaactccttgaactcctaaggccagga
gttcaagaccagcctggataacatagcaagaccccatctctacaaaaaa
ccaaaaccaaacaaacaaaaatgatagtggtgcttccctcaggatgctt
gtggtctaatgggagacagaacagcaaagggatgattagaagttggttg
ctgtgagccaggcacagtgctgatataatcccagcgctatgggaggctg
aggtgggtggatcatttgaggccaggagtttaagaccagcctggtcaac
atggtaaaaccccatctctacttaaaaatacaaaaaagttagccaggca
tggtggcatacacctgtaacccagctactcaggaggctgaggcacatga
atcacttgaacccaggaggcagaggttgctgtgcaccactgcactccag
cctgggtgacagaacgagaccttgactcaaaaaaaaaaaaaagaagttt
gttgctatggaagggtcctactcagagcaggcaccccagttaatctcat
tcaccccacatttcacatttgaacatcatcccatagcccagagcatccc
tccactgcaaaggatttattcaacatttaaacaatcctttttactttcat
tttc
(SEQ ID NO: 2)
MNSFSTSKCRKSLALELPAAVEPCVREGCVAQGGLAGGQQQRQAPSCAV
SSPLRSLPSGTGAFGPVAFSLGLLLVLPAAFPAPVPPGEDSKDVAAPHR
QPLTSSERIDKQIRYILDGISALRKETCNKSNMCESSKEALAENNLNLP
KMAEKDGCFQSGFNEETCLVKIITGLLEFEVYLEYLQNRFESSEEQARA
VQMSTKVLIQFLQKKVGVSSFPQLGVGEDRLKDSVLDNSGMQCHFQKRR
LHVNKRV

### Standard-of-Care Therapies for Cancer

Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014).

### Pharmaceutical Compositions and Dosages

Therapeutic agents of the present invention may be constituted in a composition, e.g., a pharmaceutical composition containing an Ab and soluble IL-6 and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an Ab and/or soluble IL-6 is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-PD-1 Ab, especially in combination with another anti-cancer agent, the dosage may range from about 0.01 to about 20 mg/kg, preferably from about 0.1 to about 10 mg/kg, of the subject's body weight. For example, dosages can be 0.1, 0.3, 1, 2, 3, 5 or 10 mg/kg body weight, and more preferably, 0.3, 1, 2, 3, or 5 mg/kg body weight. The dosing schedule is typically designed to achieve exposures that result in sustained receptor occupancy (RO) based on typical pharmacokinetic properties of an Ab. An exemplary treatment regime entails administration once per week, once every 2 weeks, once every 3 weeks, once every 4 weeks, once a month, once every 3-6 months or longer. In certain preferred embodiments, an anti-PD-1 Ab such as nivolumab is administered to the subject once every 2 weeks. In other preferred embodiments, the Ab is administered once every 3 weeks. The dosage and scheduling may change during a course of treatment. For example, a dosing schedule for anti-PD-1 monotherapy may comprise administering the Ab: (i) every 2 weeks in 6-week cycles; (ii) every 4 weeks for six dosages, then every three months; (iii) every 3 weeks; (iv) 3-10 mg/kg once followed by 1 mg/kg every 2-3 weeks. Considering that an IgG4 Ab typically has a half-life of 2-3 weeks, a preferred dosage regimen for an anti-PD-1 Ab of the invention comprises 0.3-10 mg/kg body weight, preferably 1-5 mg/kg body weight, more preferably 1-3 mg/kg body weight via intravenous administration, with the Ab being given every 14-21 days in up to 6-week or 12-week cycles until complete response or confirmed progressive disease.

When used in combinations with other cancer agents, the dosage of an anti-PD-1 Ab may be lowered compared to the monotherapy dose. For example, a dosage of nivolumab that is significantly lower than the typical 3 mg/kg every 3 weeks, for instance 0.1 mg/kg or less every 3 or 4 weeks, is regarded as a subtherapeutic dosage. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., 2010). Thus, 0.3 mg/kg dosing may allow for sufficient exposure to lead to maximal biologic activity.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

For combination of nivolumab with other anti-cancer agents, these agents are preferably administered at their approved dosages. The dose of IL-6 used in the Examples was determined from literature showing that this dose range (1-10 micrograms/kg body weight) of recombinant human IL-6, administered subcutaneously 3 times per week is safe and efficacious in reducing or preventing diabetes- or chemotherapy-mediated neuropathy in rodents (Andriambeloson et al, 2006; Callizot et al, 2008). The dose of IL-6 to be administered in humans is in the range of 0.06 to 3 μg/kg body weight, preferably in the range of 0.1 to 2 μg/kg body weight. Because IL-6 is a potent pro-inflammatory cytokine it is critical to be able to use an efficacious dose of IL-6 that has been found to be safe and to not result in adverse events that would otherwise limit its use. In one embodiment, the therapeutic dosage of IL-6 is a low dosage.

Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 Ab. The anti-PD-1 Ab may be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the Ab and/or cytokine in the subject. In general, human Abs show the longest half-life, followed by humanized Abs, chimeric Abs, and nonhuman Abs. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results. In certain embodiments, the combination therapy of the present invention (e.g., administration of an anti-PD-1 antibody and another anti-cancer agent) effectively increases the duration of survival of the subject. For example, the duration of survival of the subject is increased by at least about 2 months when compared to another subject treated with only one therapy (e.g., an anti-PD-1 antibody or another anti-cancer agent). In certain embodiments, the combination therapy of the present invention (e.g., administration of an anti-PD-1 antibody and another anti-cancer agent) effectively increases the duration of progression free survival of the subject. For example, the progression free survival of the subject is increased by at least about 2 months when compared to another subject treated with only one therapy (e.g., an anti-PD-1 antibody or another anti-cancer agent). In certain embodiments, the combination therapy of the present invention (e.g., administration of an anti-PD-1 antibody and another anti-cancer agent) effectively increases the response rate in a group of subjects. For example, the response rate in a group of subjects is increased by at least 2% when compared to another group of subjects treated with only one therapy (e.g., an anti-PD-1 antibody or another anti-cancer agent).

Combination of an Anti-PD-1 Ab with IL-6 for Treating Cancer

For the combination of an anti-PD-1 and IL-6, the dosing regimen may comprise an induction period (also referred to herein as an induction phase) during which one or more, preferably about four, combination doses of the anti-PD-1 and IL-6 are administered to the patient, followed by a maintenance period or phase comprising dosing with the anti-PD-1 Ab alone, i.e., not including the IL-6. In certain embodiments, the method comprises (a) an induction phase, wherein the anti-PD-1 or antigen-binding portions thereof and IL-6 are administered in combination in 2, 4, 6, 8 or 10 doses, each dose ranging from 0.1 to 10.0 mg/kg or 0.06 to 3 μg/kg body weight, respectively, administered at least once every 2 weeks, once every 3 weeks, or once every 4 weeks, followed by (b) a maintenance phase, wherein no IL-6 is administered and the anti-PD-1 antibody or antigen-binding portion thereof is repeatedly administered at a dose ranging from 0.1 to 10 mg/kg at least once every 2 weeks, once every 3 weeks, or once every 4 weeks.

Because of durability of the clinical effect previously demonstrated with immunotherapy by inhibition of immune checkpoints (see, e.g., WO 2013/173223), the maintenance phase may include, in alternative embodiments, a finite number of doses, e.g., 1-10 doses, or may involve dosing at long intervals, e.g., once every 3-6 months or once every 1-2 years or longer intervals. The maintenance phase may be continued for as long as clinical benefit is observed or until unmanageable toxicity or disease progression occurs. In certain preferred embodiments of the present methods, the anti-PD-1 Ab is nivolumab.

Certain preferred embodiments of the present methods comprise (a) an induction phase consisting of administration of nivolumab by intravenous infusion followed by administration of IL-6 by intravenous infusion every 3 weeks for 4 combination doses, followed by (b) maintenance dosing with nivolumab administered by intravenous infusion every 2 weeks starting 3 weeks after the 4th dose of induction therapy or after Day 113 if the 4th dose of induction therapy has not been administered due to treatment delays.

In certain embodiments, the anti-PD-1 Ab or antigen-binding portion thereof is administered at a subtherapeutic dose. In certain other embodiments, the IL-6 is administered at a subtherapeutic dose. In further embodiments, both the anti-PD-1 Ab or antigen-binding portion thereof and the IL-6 are each administered at a subtherapeutic dose.

### Kits

Also within the scope of the present invention are kits comprising an anti-PD-1 Ab and soluble IL-6 for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a lung cancer, the kit comprising: (a) a dosage ranging from 0.1 to 10 mg/kg body weight of an anti-cancer agent which is an Ab or an antigen-binding portion thereof that specifically binds to the PD-1 receptor and inhibits PD-1 activity; (b) a dosage of soluble IL-6 which is a dosage ranging from 0.06 to 3 μg/kg body weight; and (c) instructions for using the anti-PD-1 Ab and soluble IL-6 in any of the combination therapy methods disclosed herein. In certain embodiments, the anti-PD-1Ab and/or the IL-6 may be co-packaged in unit dosage form. In certain preferred embodiments for treating human patients, the kit comprises an anti-human PD-1 Ab disclosed herein, e.g., nivolumab or pembrolizumab. In other preferred embodiments, the kit comprises IL-6.

### Example 1

### Recombinant Human (rh)IL-6 and Anti-Mouse PD-1 Monoclonal Antibody (mAb) in a Mouse Subcutaneous CT-26 Colon Carcinoma Model

### Materials and Methods

### Animals:

Female BALB/c mice (Harlan Laboratories, Livermore, Calif.) were acclimated for a minimum of three days prior to the start of the studies. Mice were housed 4-5 animals per cage, and the cages were placed in microisolator ventilated racks. Housing was at 18-26° C. and 50±20% relative humidity with at least twelve room air changes per hour. A 12h light/dark cycle was maintained. Animals were provided with sanitized laboratory rodent diet and municipal water ad libitum.

### Preparation and Implantation of Tumor Cells

CT-26 cells were obtained from the BMS master cell bank and maintained in RPMI-1640 medium (Hyclone, Cat. No. SH30096.01) supplemented with 10% fetal bovine serum (FBS; Hyclone, Cat. No. SH30071.03). Approximately twice a week, cells contained in a single T175 flask were divided and expanded to four T175 flasks at a 1:5 dilution until sufficient number of cells were obtained for tumor implantation. The cells were harvested near 80% confluence, washed and resuspended in PBS (Hyclone, Cat. No. SH30256.01).

On Day 0, 1×10⁶ CT-26 cells were implanted into the mice using a 1 cc syringe (Becton Dickinson, Franklin Lakes, N.J.) and 27 gauge ⅝ inch needle. Tumors were then measured two times weekly in 3 dimensions with an electronic caliper (Mitutoyo, Aurora, Ill.) and recorded. Tumor volumes (mm³) were calculated using the formula: width×length×height×0.5.

### IL-6 and Antibody Treatments

Following tumor volume measurements on Day 7 post-tumor implantation, mice were staged according to tumor volume. Mice with a mean tumor volume of 23 mm³ were randomized into groups and treated as shown in Table 1.

The mouse (m)IgG1 isotype control antibody is an inert monoclonal antibody (mAb) obtained from a mouse hybridoma against glu-glu. It was prepared in PBS immediately prior to administration to provide doses of 10 mg/kg per mouse via intraperitoneal (IP) injection on Days 7, 10 and 13 as shown in Table 1.

The monoclonal antibody against mouse PD-1 (clone 4H2) was prepared in PBS immediately prior to administration to provide doses of 10 mg/kg per mouse via intraperitoneal (IP) injection on Days 7, 10 and 13 as shown in Table 1. This antibody was produced at Bristol-Myers Squibb and was a mouse IgG1 that had been engineered to not bind Fc receptors.

Recombinant human IL-6 (rhIL-6) (R&D Systems, carrier-free, Cat. No. 206-IL-200/CF) was prepared in 0.02% (w/v) mouse albumin (Sigma-Aldrich, Cat. No. A3139) immediately prior to administration via subcutaneous (SC) injection at doses of 10 micrograms/kg, 3 times per week, beginning on Day 7 as shown in Table 1.

TABLE 1
Experiment #1 Treatment Groups

| Treatment | N | Route | Dose | Treatment schedule |
|---|---|---|---|---|
| mIgG1 control | 10 | IP | 10 mg/kg | d. 7, 10, 13 |
| mPD-1 mAb | 10 | IP | 10 mg/kg | d. 7, 10, 13 |
| hIL-6 | 9 | SC | 10 μg/kg | d. 7, 10, 13, 15, 17, |
| | | | | 20, 22, 24, 27, 29, 31 |
| hIL-6 | 9 | SC | 10 μg/kg | hIL-6: d. 7, 10, 13, |
| | | | hIL-6 | 15, 17, 20, 22, 24, |
| | | | | 27, 29, 31 |
| PD-1 mAb | | IP | 10 mg/kg | mPD-1 mAb: d. 7, 10, 13 |
| | | | mPD-1 mAb | |

h = human;
m = mouse-specific

### Post-Treatment Monitoring

Animals were checked daily for postural, grooming, and respiratory changes, as well as lethargy. Animals were weighed two times weekly and euthanized if weight loss was ≥20%.

Mice were checked for the presence and size of tumors twice weekly until death or euthanasia. Tumors were measured in 3 dimensions with an electronic caliper (Mitutoyo, Aurora, Ill.) and recorded. Response to treatment compounds was measured as a function of tumor growth. If the tumor reached a volume of ≥1500 mm³ or appeared ulcerated, animals were euthanized.

### Statistical Analyses

Statistical analyses were performed using GraphPad Prism software (Version 5.01), with the statistical test that was utilized being noted in the figure legends. A p value <0.05 was considered statistically significant. Mean and median tumor volumes for groups were calculated while at least 70% of study animals remained alive.

### Results

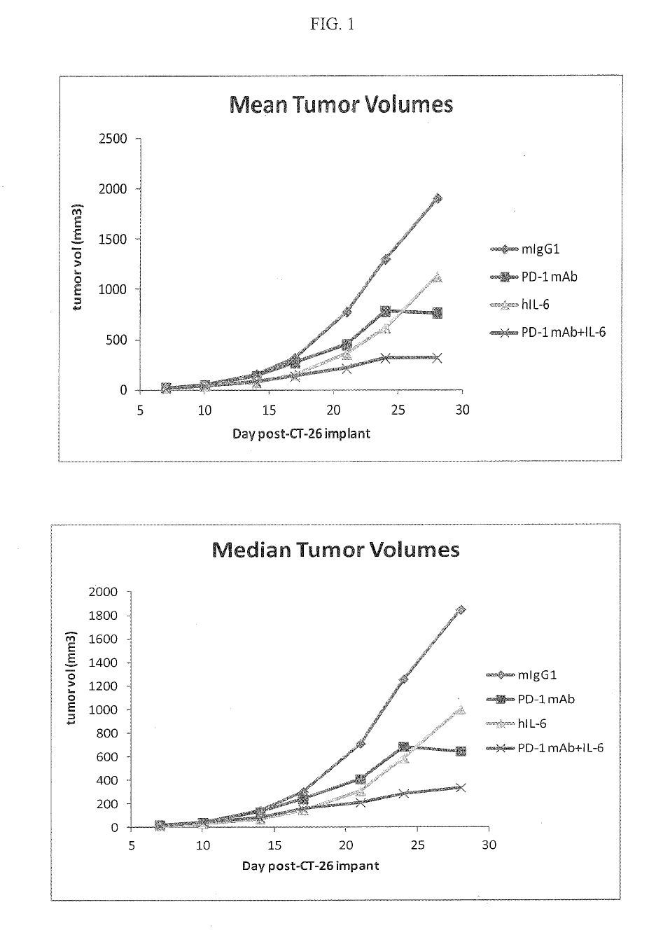

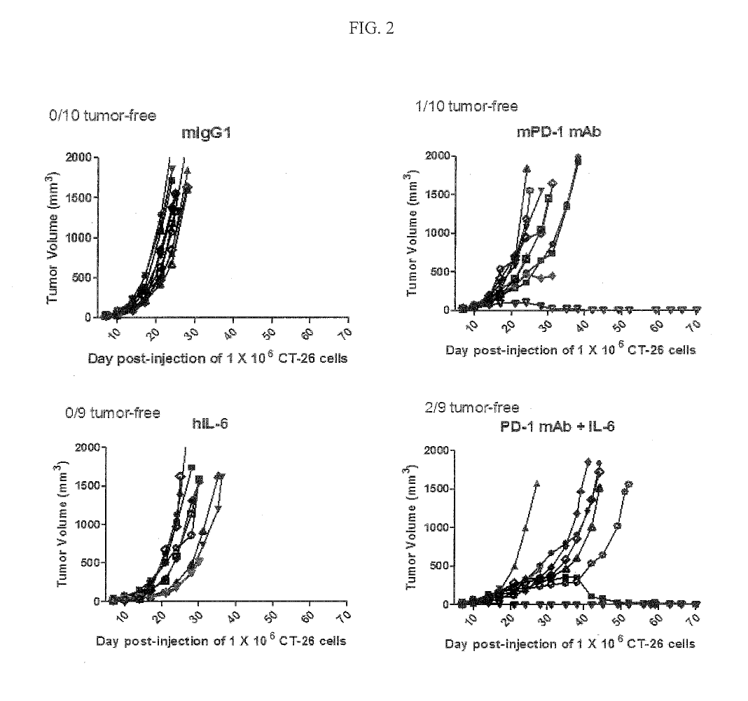

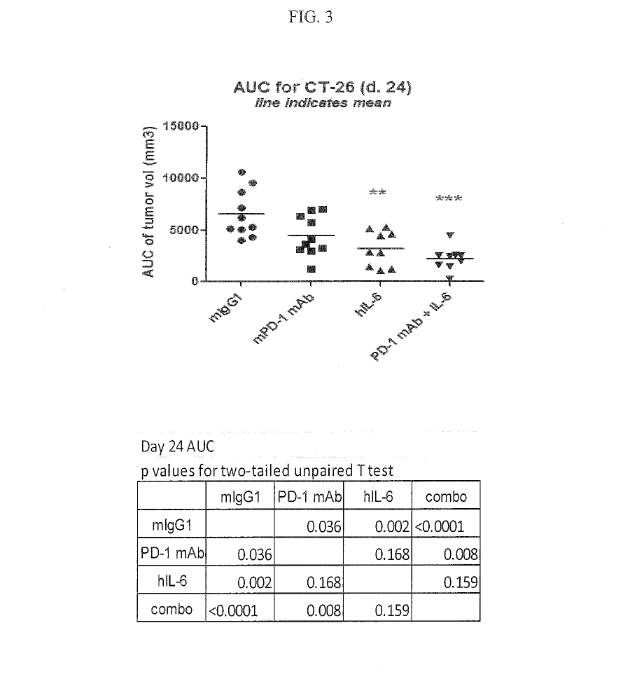

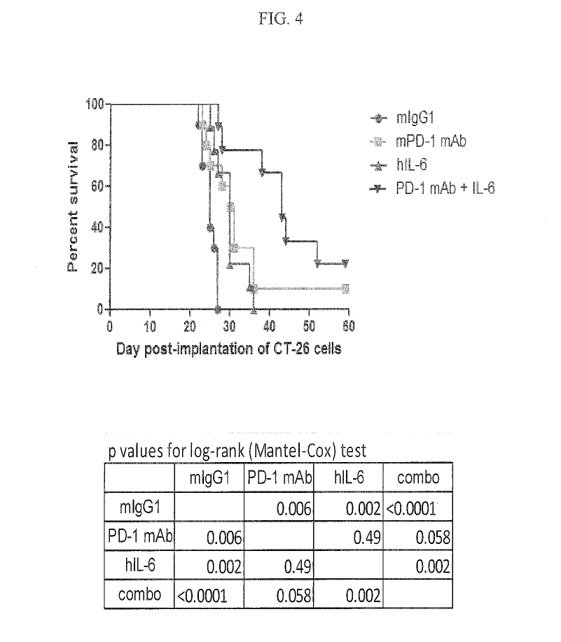

Mice treated with mPD-1 mAb or hIL-6 as single agents had some tumor growth delays as compared to the mIgG1 group, with significantly lower means (p<0.05 or better by two-way ANOVA; FIG. 1) and either delays in individual tumor volumes, or in the case of PD-1 mAb, 1/10 (10%) tumor-free mice (FIG. 2). On the other hand, the combination of mPD-1 mAb and hIL-6 led to greater tumor growth inhibition than either treatment alone, and 2/9 (22%) tumor-free mice. This can also be demonstrated by analyzing the area-under-the-curve (AUC) for each of the tumor growths of individual mice which in this case, through Day 24 (the latest day possible given the loss of mice due to tumor burden and/or ulceration), hIL-6 treatment resulted in statistically significantly lower (p<0.01) mean AUC values as compared to mIgG1 treated mice (FIG. 3). Importantly, combination treatment with mPD-1 mAb+hIL-6 resulted in even lower mean AUC values (p<0.001 vs. mIgG1-treated mice). An additional way to analyze the data from this study is by survival analysis, an important factor to consider with potential antitumor therapeutics. As shown in FIG. 4, mPD-1 mAb and hIL-6 each were able to significantly enhance survival as single agents compared to mIgG1-treated mice (p=0.006 and p=0.002, respectively), whereas the group of mice treated with the combination of mPD-1 mAb+hIL-6 had the greatest prolongation of survival (p<0.0001 vs. mIgG1 group; p=0.058 vs. mPD-1 group; p=0.002 vs. hIL-6 group).

FIG. 1 shows the mean and median tumor volumes by group showing antitumor activity of mPD-1 mAb and hIL-6, alone or in combination, in the CT-26 tumor model. Mean and median tumor volumes are shown for groups up until all groups had ≥70% living. Two-way (repeated-measures) ANOVA analyses were performed on Day 24 which was the latest day possible due to the loss of mice as a result of tumor burden or tumor ulceration: p=0.032 for PD-1 mAb vs. mIgG1 by two-way (repeated measures) ANOVA for treatment. p=0.002 for hIL-6 vs. mIgG1 by two-way (repeated measures) ANOVA for treatment. p<0.0001 for PD-1 mAb+hIL-6 combination vs. mIgG1 by two-way (repeated measures) ANOVA for treatment.

FIG. 2 shows individual mouse tumor volume data showing antitumor activity of mPD-1 mAb and hIL-6, alone or in combination, in the CT-26 tumor model. Each line represents an individual mouse. Red data lines note that the mouse was euthanized due to tumor ulceration.

FIG. 3 shows the area-under-the-curve (AUC) values for tumor volumes through Day 24 showing antitumor activity of mPD-1 mAb and hIL-6, alone or in combination, in the CT-26 tumor model. Statistical analyses between all groups was conducted using one-way ANOVA (p=0.0001), followed by Dunn's multiple comparison tests between groups: ** p<0.01 for hIL-6 vs. mIgG1, and ***p<0.001 for mPD-1 mAb+hIL-6 combination vs. mIgG1. Two-tailed unpaired T tests were conducted to analyze differences between two groups (shown in the insert).

FIG. 4 shows survival proportions for mice treated with mIgG1, or mPD-1 mAb or hIL-6 alone and in combination in the CT-26 tumor model. Death day was designated when the tumor volume reached 1500 mm³ or the mouse was euthanized due to an ulcerated tumor. Overall log-rank analysis (via Mantel-Cox test): p=0.0002; log-rank for trend: p=0.0004. The p values for log-rank/Mantel-Cox analyses between individual groups are shown in the insert.

### Example 2

### Recombinant Human IL-6 and Anti-Mouse PD-1 Monoclonal Antibody in a Mouse Subcutaneous MC-38 Adenocarcinoma Model

### Materials and Methods

### Animals:

Female C57BL/6 mice (Harlan Laboratories, Livermore, Calif.) were acclimated for a minimum of three days prior to the start of the studies. Mice were housed 4-5 animals per cage, and the cages were placed in microisolator ventilated racks. Housing was at 18-26° C. and 50±20% relative humidity with at least twelve room air changes per hour. A 12 h light/dark cycle was maintained. Animals were provided with sanitized laboratory rodent diet and municipal water ad libitum.

### Preparation and Implantation of Tumor Cells

MC-38 cells were obtained from the BMS master cell bank and maintained in Dulbecco's modified Eagle's medium (DMEM; Hyclone, Cat. No. SH30081.01) supplemented with 10% fetal bovine serum (FBS; Hyclone, Cat. No. SH30071.03). Approximately twice a week, cells contained in a single T175 flask were divided and expanded to four T175 flasks at a 1:5 dilution until sufficient number of cells were obtained for tumor implantation. The cells were harvested near 80% confluence, washed and resuspended in PBS (Hyclone, Cat. No. SH30256.01).

On Day 0, 0.5×10⁶ MC-38 cells were implanted into the mice using a 1 cc syringe (Becton Dickinson, Franklin Lakes, N.J.) and 27 gauge ⅝ inch needle. Tumors were then measured two times weekly in 3 dimensions with an electronic caliper (Mitutoyo, Aurora, Ill.) and recorded. Tumor volumes (mm³) were calculated using the formula: width×length×height×0.5.

### IL-6 and Antibody Treatments

Following tumor volume measurements on Day 8 post-tumor implantation, mice were staged according to tumor volume. Mice with a mean tumor volume of 33 mm³ were randomized into groups and treated as shown in Table 2.

The mouse (m)IgG1 isotype control antibody is an inert monoclonal antibody (mAb) obtained from a mouse hybridoma against glu-glu. It was prepared in PBS immediately prior to administration to provide doses of 20 mg/kg for the mIgG1 group, or as a 10 mg/kg supplement to the mPD-1 mAb groups which ensured that all antibody-containing groups received a total of 20 mg/kg IgG. The antibody was delivered via intraperitoneal (IP) injection on Days 8, 11 and 14 as shown in Table 2.

The monoclonal antibody against mouse PD-1 (clone 4H2) was prepared in PBS immediately prior to administration to provide doses of 10 mg/kg per mouse via intraperitoneal (IP) injection on Days 8, 11 and 14 as shown in Table 2. This antibody was produced at Bristol-Myers Squibb and was a mouse IgG1 that had been engineered to not bind Fc receptors.

Recombinant human IL-6 (rhIL-6) (R&D Systems, carrier-free, Cat. No. 206-IL-200/CF) was prepared in 0.02% (w/v) mouse albumin (Sigma-Aldrich, Cat. No. A3139) immediately prior to administration via subcutaneous (SC) injection at doses of 10 micrograms/kg, 3 times per week, beginning on Day 8 as shown in Table 2.

TABLE 2
Experiment #2 Treatment Groups

| Treatment | N | Route | Dose | Treatment schedule |
|---|---|---|---|---|
| mIgG1 control | 9 | IP | 20 mg/kg | d. 8, 11, 14 |
| mPD-1 mAb | 9 | IP | 10 mg/kg mIgG1 | d. 8, 11, 14 |
| | | | control + 10 | |
| | | | mg/kg mPD-1 mAb | |
| hIL-6 | 9 | SC | 10 μg/kg hIL-6 | d. 8, 10, 13, 15, |
| | | | | 17, 20, 22, 24, |
| | | | | 27, 29, 31 |
| hIL-6 | 9 | SC | 10 μg/kg hIL-6 | hIL-6: d. 8, 10, 13, |
| | | | | 15, 17, 20, 22, 24, |
| | | | | 27, 29, 31 |
| PD-1 mAb | | IP | 10 mg/kg mIgG1 | mPD-1 mAb: d. 8, 11, |
| | | | control + 10 | 14 |
| | | | mg/kg mPD-1 mAb | |

h = human;
m = mouse-specific

### Post-Treatment Monitoring

Animals were checked daily for postural, grooming, and respiratory changes, as well as lethargy. Animals were weighed two times weekly and euthanized if weight loss was ≥20%.

Mice were checked for the presence and size of tumors twice weekly until death or euthanasia. Tumors were measured in 3 dimensions with an electronic caliper (Mitutoyo, Aurora, Ill.) and recorded. Response to treatment compounds was measured as a function of tumor growth. If the tumor reached a volume of ≥1500 mm³ or appeared ulcerated, animals were euthanized.

### Statistical Analyses

Statistical analyses were performed using GraphPad Prism software (Version 5.01), with the statistical test that was utilized being noted in the figure legends. A p value <0.05 was considered statistically significant. Mean and median tumor volumes for groups were calculated while at least 70% of study animals remained alive.

### Results

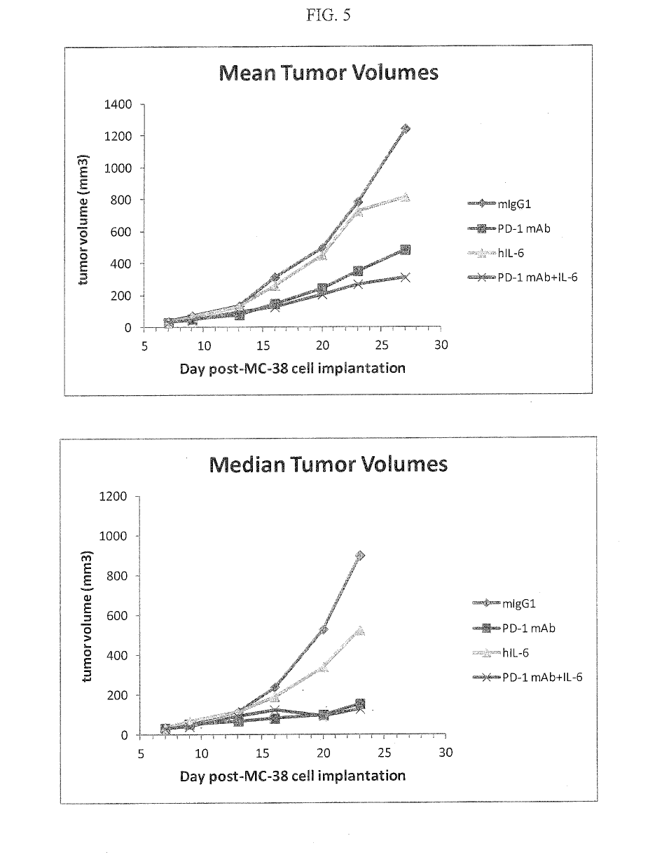

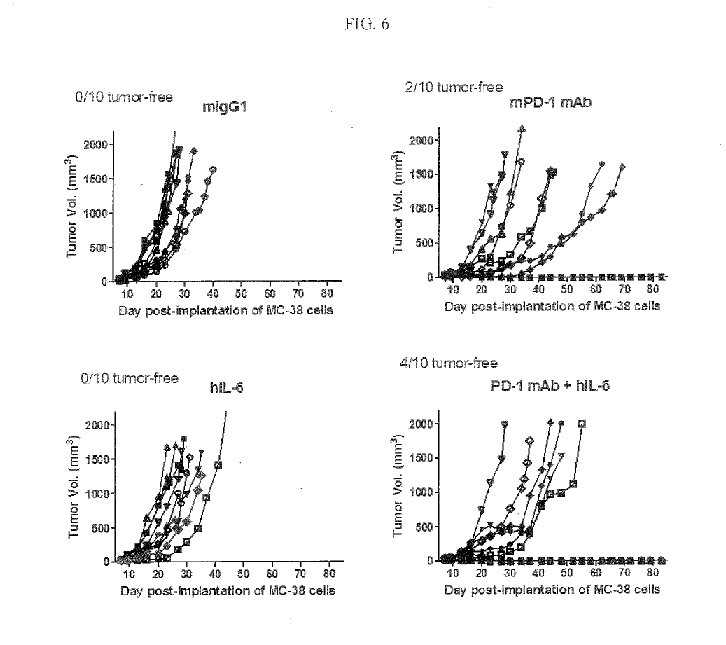

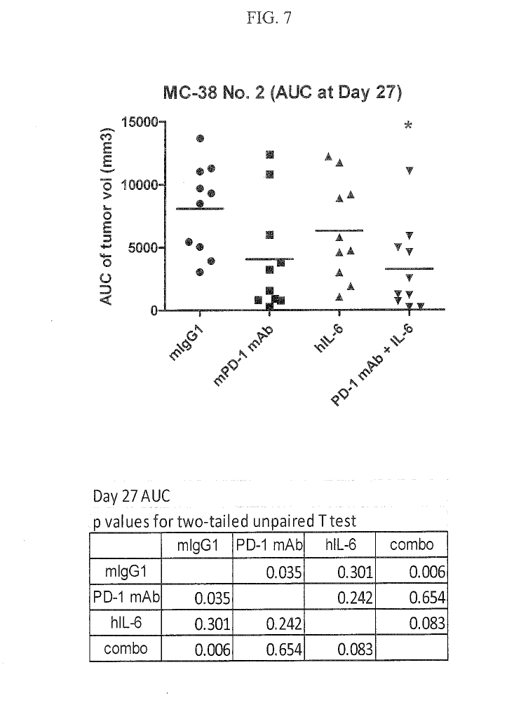

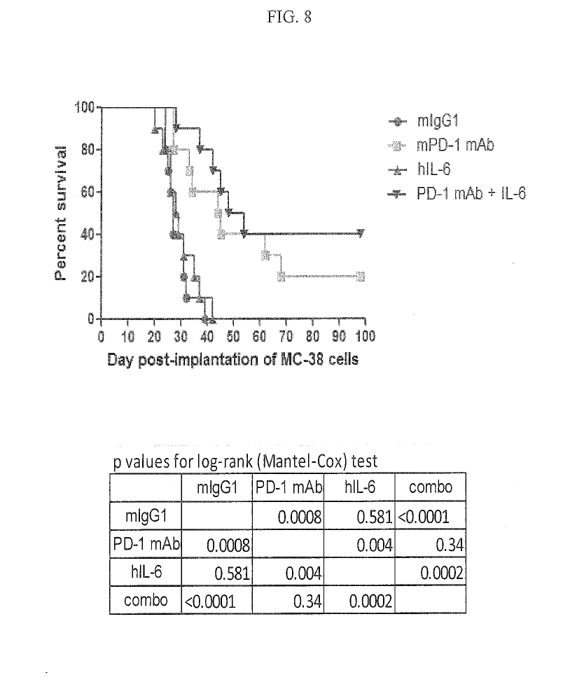

Mice treated with mPD-1 mAb or hIL-6 as single agents had some tumor growth delays as compared to the mIgG1 group, with significantly lower means (p<0.05 or better by two-way ANOVA; FIG. 5) and either delays in individual tumor volumes, or in the case of PD-1 mAb, 2/10 (20%) tumor-free mice (FIG. 6). On the other hand, the combination of mPD-1 mAb and hIL-6 led to greater tumor growth inhibition than either treatment alone, and 4/10 (40%) tumor-free mice. This can also be demonstrated by analyzing the area-under-the-curve (AUC) for each of the tumor growths of individual mice which in this case, through Day 27 (the latest day possible given the loss of mice due to tumor burden and/or ulceration), combination treatment with mPD-1 mAb+hIL-6 resulted in significantly lower mean AUC values (p<0.05 vs. mIgG1-treated mice) (FIG. 7). An additional way to analyze the data from this study is by survival analysis, an important factor to consider with potential antitumor therapeutics. As shown in FIG. 8, mPD-1 mAb was able to significantly enhance survival as single agents compared to mIgG1-treated mice (p=0.0008), whereas the group of mice treated with the combination of mPD-1 mAb+hIL-6 had an even greater prolongation of survival (p<0.0001 vs. mIgG1 group; p=0.32 vs. mPD-1 group; p=0.0002 vs. hIL-6 group).

This experiment was conducted two times, with similar results for both. The combination efficacy observed in this additional well-established pre-clinical tumor model suggests that co-treatment with PD-1 mAb and recombinant IL-6 could be an effective method for the treatment of cancer.

FIG. 5 shows the mean and median tumor volumes by group showing antitumor activity of mPD-1 mAb and hIL-6, alone or in combination, in the MC-38 tumor model. Mean and median tumor volumes are shown for groups up until all groups had ≥70% living. Two-way (repeated-measures) ANOVA analyses were performed on Day 23 which was the latest day possible due to the loss of mice as a result of tumor burden or tumor ulceration: p=0.035 for PD-1 mAb vs. mIgG1 by two-way (repeated measures) ANOVA for treatment. p=0.009 for PD-1 mAb+hIL-6 combination vs. mIgG1 by two-way (repeated measures) ANOVA for treatment.

FIG. 6 shows individual mouse tumor volume data showing antitumor activity of mPD-1 mAb and hIL-6, alone or in combination, in the MC-38 tumor model. Each line represents an individual mouse. Red data lines note that the mouse was euthanized due to tumor ulceration.

FIG. 7 shows the area-under-the-curve (AUC) values for tumor volumes through Day 27 showing antitumor activity of mPD-1 mAb and hIL-6, alone or in combination, in the MC-38 tumor model. Day 27 was the latest day possible to analyze due to the loss of mice from tumor burden or tumor ulceration. Statistical analyses between all groups were conducted using one-way ANOVA (p=0.032), followed by Dunn's multiple comparison tests between groups: * p<0.05 for mPD-1 mAb+hIL-6 combination vs. mIgG1. Two-tailed unpaired T tests were conducted to analyze differences between two groups (shown in the insert).

FIG. 8 shows the survival proportions for mice treated with mIgG1, or mPD-1 mAb or hIL-6 alone and in combination in the MC-38 tumor model. Death day was designated when the tumor volume reached 1500 mm³ or the mouse was euthanized due to an ulcerated tumor. Overall log-rank analysis (via Mantel-Cox test): p=0.001; log-rank for trend: p=0.0099. Thep values for log-rank/Mantel-Cox analyses between individual groups are shown in the insert.

### Example 3

### Recombinant Mouse IL-6 and Anti-Mouse PD-1 Monoclonal Antibody (mAb) in a Mouse Subcutaneous CT-26 Colon Carcinoma Model

### Materials and Methods

### Animals:

Female BALB/c mice (Harlan Laboratories, Livermore, Calif.) were acclimated for a minimum of three days prior to the start of the studies. Mice were housed 5 animals per cage, and the cages were placed in microisolator ventilated racks. Housing was at 18-26° C. and 50±20% relative humidity with at least twelve room air changes per hour. A 12 h light/dark cycle was maintained. Animals were provided with sanitized laboratory rodent diet and municipal water ad libitum.

### Preparation and Implantation of Tumor Cells

CT-26 cells were obtained from the BMS master cell bank and maintained in RPMI-1640 medium (Hyclone, Cat. No. SH30096.01) supplemented with 10% fetal bovine serum (FBS; Hyclone, Cat. No. SH30071.03). Approximately twice a week, cells contained in a single T175 flask were divided and expanded to four T175 flasks at a 1:5 dilution until sufficient number of cells were obtained for tumor implantation. The cells were harvested near 80% confluence, washed and resuspended in PBS (Hyclone, Cat. No. SH30256.01).

On Day 0, 1×10⁶ CT-26 cells were implanted into the mice using a 1 cc syringe (Becton Dickinson, Franklin Lakes, N.J.) and 27 gauge ⅝ inch needle. Tumors were then measured two times weekly in 3 dimensions with an electronic caliper (Mitutoyo, Aurora, Ill.) and recorded. Tumor volumes (mm³) were calculated using the formula: width×length×height×0.5.

### IL-6 and Antibody Treatments

Following tumor volume measurements on Day 7 post-tumor implantation, mice were staged according to tumor volume. Mice with a mean tumor volume of 26 mm³ were randomized into groups and treated as shown in Table 3.

The mouse (m)IgG1 isotype control antibody is an inert monoclonal antibody (mAb) obtained from a mouse hybridoma against glu-glu. It was prepared in PBS immediately prior to administration to provide doses of 10 mg/kg per mouse via intraperitoneal (IP) injection on Days 7, 10 and 13 as shown in Table 3.

The monoclonal antibody against mouse PD-1 (clone 4H2) was prepared in PBS immediately prior to administration to provide doses of 10 mg/kg per mouse via intraperitoneal (IP) injection on Days 7, 10 and 13 as shown in Table 3. This antibody was produced at Bristol-Myers Squibb and was a mouse IgG1 that had been engineered to not bind Fc receptors.

Recombinant mouse IL-6 (mIL-6) (R&D Systems, carrier-free, Cat. No. 406-ML-025/CF) was prepared in 0.02% (w/v) mouse albumin (Sigma-Aldrich, Cat. No. A3139) immediately prior to administration via subcutaneous (SC) injection at doses of 3 micrograms/kg, 3 times per week, beginning on Day 7 as shown in Table 3. The dose of mIL-6 was calculated from the 3-10-fold greater activity that the mIL-6 was shown to have as compared to the hIL-6 on mouse plasmacytoma cells (assay described in Nordan R P et al, 1987).

TABLE 3
Experiment #3 Treatment Groups

| Treatment | N | Route | Dose | Treatment schedule |
|---|---|---|---|---|
| mIgG1 control | 10 | IP | 10 mg/kg | d. 7, 10, 13 |
| mPD-1 mAb | 10 | IP | 10 mg/kg | d. 7, 10, 13 |
| mIL-6 | 10 | SC | 3 μg/kg | d. 7, 10, 13, 15, |
| | | | | 17, 20, 22, 24, 27, |
| | | | | 29, 31 |
| mIL-6 | 10 | SC | 3 μg/kg | mIL-6: d. 7, 10, 13, |
| | | | mIL-6 | 15, 17, 20, 22, 24, |
| | | | | 27, 29, 31 |
| PD-1 mAb | | IP | 10 mg/kg | mPD-1 mAb: d. 7, 10, |
| | | | mPD-1 mAb | 13 |

m = mouse-specific

### Post-Treatment Monitoring

Animals were checked daily for postural, grooming, and respiratory changes, as well as lethargy. Animals were weighed two times weekly and euthanized if weight loss was ≥20%.

Mice were checked for the presence and size of tumors twice weekly until death or euthanasia. Tumors were measured in 3 dimensions with an electronic caliper (Mitutoyo, Aurora, Ill.) and recorded. Response to treatment compounds was measured as a function of tumor growth. If the tumor reached a volume of ≥1500 mm³ or appeared ulcerated, animals were euthanized.

### Statistical Analyses

Statistical analyses were performed using GraphPad Prism software (Version 5.01), with the statistical test that was utilized being noted in the figure legends. A p value <0.05 was considered statistically significant. Mean and median tumor volumes for groups were calculated while at least 70% of study animals remained alive.

### Results

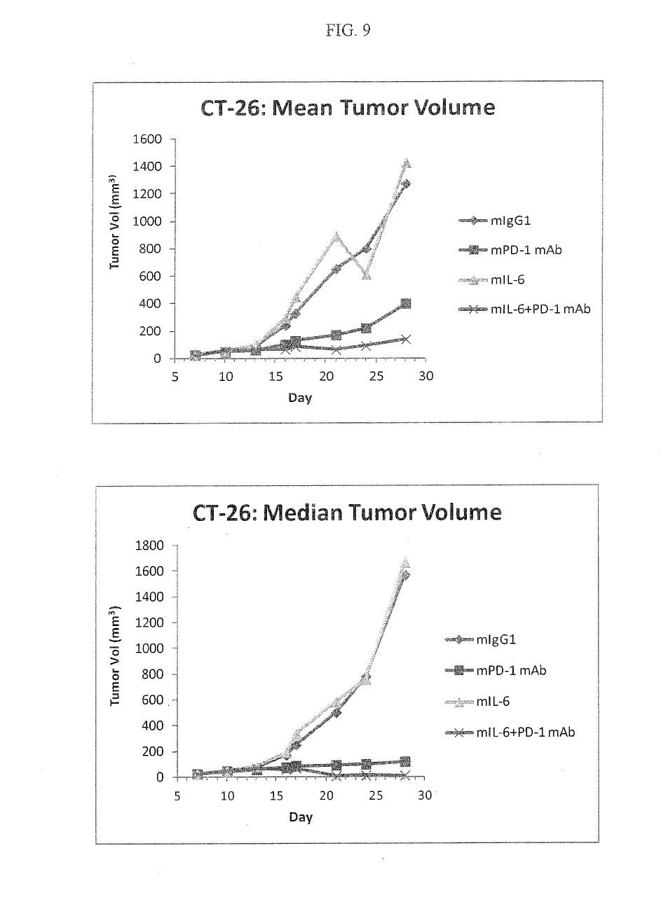

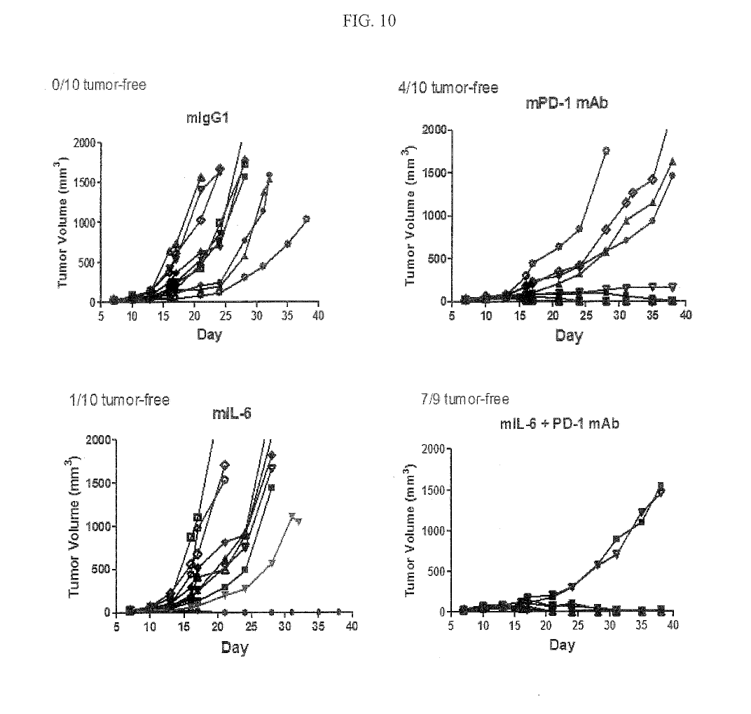

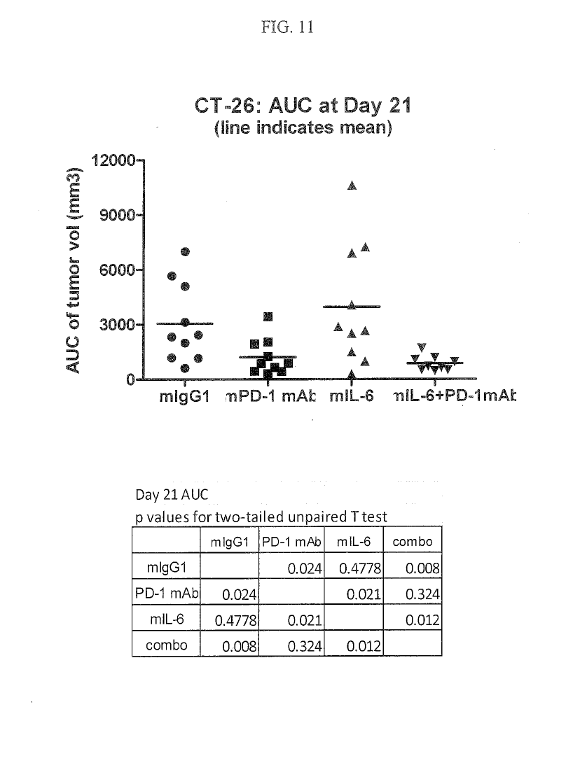

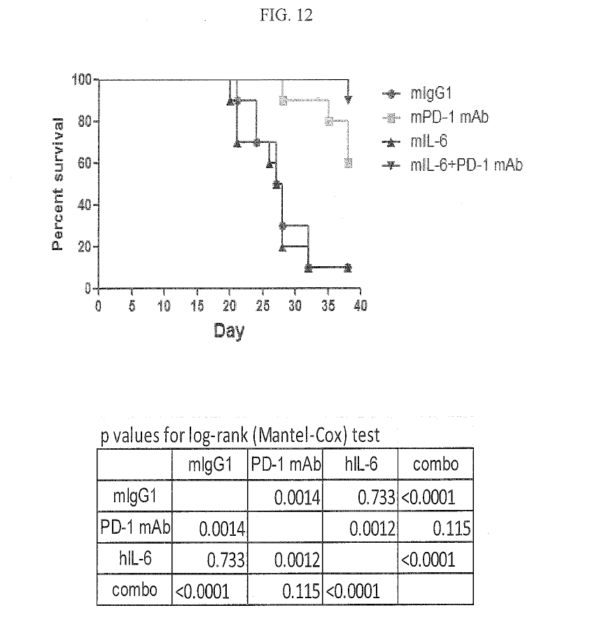

Mice treated with mPD-1 mAb or mIL-6 as single agents had some tumor growth delays as compared to the mIgG1 group, with a significantly lower mean for mPD-1 mAb treated mice (p=0.023 by two-way ANOVA; FIG. 9) and either delays in individual tumor volumes or a greater number of tumor-free mice (FIG. 10). In this case, mPD-1 mAb treatment resulted in 4/10 (40%) tumor-free mice and mIL-6 treatment resulted in 1/10 (10%) tumor-free mice. However, the combination of mPD-1 mAb and mIL-6 led to greater tumor growth inhibition than either treatment alone with 7/9 (78%) tumor-free mice. This can also be demonstrated by analyzing the area-under-the-curve (AUC) for each of the tumor growths of individual mice which in this case, through Day 21 (the latest day possible given the loss of mice due to tumor burden and/or ulceration), mPD-1 mAb treatment resulted in a statistically significantly lower (p<0.05) mean AUC value as compared to mIL-6 treated mice (FIG. 11). Combination treatment with mPD-1 mAb+mIL-6 also resulted in a lower mean AUC value as compared to mIL-6-treated mice (p<0.05). As noted in Experiments 1 and 2, an additional way to analyze the data from this study is by survival analysis, an important factor to consider with potential antitumor therapeutics. As shown in FIG. 12, mPD-1 mAb was able to significantly enhance survival as a single agent compared to mIgG1-treated mice (p=0.0014), whereas the group of mice treated with the combination of mPD-1 mAb+mIL-6 had the greatest prolongation of survival (p<0.0001 vs. mIgG1 group; p=0.115 vs. mPD-1 group; p<0.0001 vs. mIL-6 group).

The combination efficacy observed in this well-established pre-clinical tumor model further supports the idea that co-treatment with PD-1 mAb and recombinant IL-6 could be an effective method for the treatment of cancer.

FIG. 9 shows the mean and median tumor volumes by group showing antitumor activity of mPD-1 mAb and mIL-6, alone or in combination, in the CT-26 tumor model. Mean and median tumor volumes are shown for groups up until all groups had ≥70% living. Two-way (repeated-measures) ANOVA analyses were performed on Day 21 which was the latest day possible due to the loss of mice as a result of tumor burden or tumor ulceration: p=0.023 for PD-1 mAb vs. mIgG1 by two-way (repeated measures) ANOVA for treatment. p=0.021 for mPD-1 mAb vs. mIL-6 by two-way (repeated measures) ANOVA for treatment. p=0.012 for mIL-6 vs. mPD-1 mAb+mIL-6 combination by two-way (repeated measures) ANOVA for treatment. p=0.007 for PD-1 mAb+mIL-6 combination vs. mIgG1 by two-way (repeated measures) ANOVA for treatment.

FIG. 10 shows the individual mouse tumor volume data showing antitumor activity of mPD-1 mAb and mIL-6, alone or in combination, in the CT-26 tumor model. Each line represents an individual mouse. Red data lines note that the mouse was euthanized due to tumor ulceration.

FIG. 11 shows the area-under-the-curve (AUC) values for tumor volumes through Day 21 showing antitumor activity of mPD-1 mAb and mIL-6, alone or in combination, in the CT-26 tumor model. Day 21 was the latest day possible to analyze due to the loss of mice from tumor burden or tumor ulceration. Statistical analyses between all groups were conducted using one-way ANOVA (p=0.0055), followed by Dunn's multiple comparison tests between groups: p<0.05 for mPD-1 mAb vs. mIL-6; p<0.05 for mIL-6 vs. mPD-1 mAb+mIL-6 combination. Two-tailed unpaired T tests were conducted to analyze differences between two groups (shown in the insert).

FIG. 12 shows the survival proportions for mice treated with mIgG1, or mPD-1 mAb or mIL-6 alone and in combination in the CT-26 tumor model. Death day was designated when the tumor volume reached 1500 mm³ or the mouse was euthanized due to an ulcerated tumor. Overall log-rank analysis (via Mantel-Cox test): p<0.0001; log-rank for trend: p=0.011. The p values for log-rank/Mantel-Cox analyses between individual groups are shown in the insert.

Taken together, the results from these studies demonstrate that co-treatment with PD-1 blockade and recombinant IL-6 is more efficacious than either treatment alone in mouse models of cancer, and support the idea that PD-1 blockade in combination with recombinant human IL-6 may be an effective treatment of cancer in humans.

### Example 4

### Immunohistochemical Analysis of Several Biomarkers in MC-38 Adenocarcinoma Model

### Materials and Methods

### Study Design:

Female C57BL/6 mice (Harlan, Livermore, Calif.) were implanted with 5×10⁵ MC-38 cells, subcutaneously on Day 0. Tumors were staged on Day 7 (mean volume=37 mm³). Treatments began Day 7. Tumors and body weight were measured twice weekly. On Day 15, tumors from all mice in all groups collected, frozen in OCT and stored at −80° C. until evaluated by IHC.

TABLE 4
Summary of Study Design

| | | Route of | | |
|---|---|---|---|---|
| | | Admin- | | Treatment |
| Group | N | istration | Dose | schedule |
| mIgG1 | 3 | IP | 10 mg/kg isotype control | d. 7, 10, 14 |
| mPD-1 mAb | 2 | IP | 10 mg/kg mAb | d. 7, 10, 14 |
| hIL-6 | 3 | SC | 10 μg/kg | d. 7, 9, 12, 14 |
| hIL-6 | 3 | SC | 10 μg/kg | d. 7, 9, 12, 14 |
| mPD-1 mAb | | IP | 10 mg/kg mAb | d. 7, 10, 14 |

### Results

MC-38 tumors showed more PD-L1 expression (primarily in the tumor periphery) and decreased vascularity (via CD31 staining) compared to the CT-26 tumors. hIL-6 treatment was associated with greater PD-L1 staining. In both tumors, CD3 staining correlated with PD-L1 expression. The combination treatment group (anti-mouse PD-1 antibody (mPD-1 mAb) and IL-6) in the CT-26 study had a significant number of CD3+ cells within the tumor itself. The histology group will next stain the tumors for CD11b, Gr-1 to look at MDSCs.

PD-L1 expression
IgG1: some expression
mPD-1 mAb: <expression than IgG1 group
hIL-6: 2/3 had >PD-L1 expression than IgG1 group
Combo: 2/3 had >PD-L1 expression than IgG1 group

CD3 expression: similar staining as PD-L1

Foxp3 expression
IgG1: low # throughout tumor
mPD-1 mAb, IL-6 and combo had low # within tumor but >common or numerous at the periphery (variable expression)

CD31 expression
IgG1: moderate # of vessels within the tumor
mPD-1 mAb: moderate # of vessels within the tumor
hIL-6: 1/3 had <# of vessels within the tumor
Combo: 3/3 had <# of vessels within the tumor

### Example 5

### Immunohistochemical Analysis of Several Biomarkers in CT-26 Adenocarcinoma Model Materials and Methods

Female BALB/c mice (Harlan, Livermore, Calif.) were implanted with 1×10⁶ CT-26 cells, subcutaneously on Day 0. Tumors were staged on Day 7 (mean volume=19 mm³). Treatments began Day 7. Tumors and body weight were measured twice weekly. On Day 14 tumors from all mice in all groups collected, frozen in OCT and stored at −80° C. until evaluated by IHC.

TABLE 5
Experiment #5 Treatment Groups

| | | Route of | | |
|---|---|---|---|---|
| | | Admin- | | Treatment |
| Group | N | istration | Dose | schedule |
| mIgG1 | 3 | IP | 10 mg/kg | d. 7, 10, 13 |
| mPD-1 mAb | 3 | IP | 10 mg/kg | d. 7, 10, 13 |
| hIL-6 | 3 | SC | 10 μg/kg | d. 7, 9, 12 |
| hIL-6 | 3 | SC | 10 μg/kg | d. 7, 9, 12 |
| mPD-1 mAb | | IP | 10 mg/kg | d. 7, 10, 13 |

PD-L1 expression
Not as increased as in MC-38. hIL-6 group has >PD-L1 expression than IgG1 group

CD3 expression
IgG1: some staining throughout tumor of varying density
mPD-1 mAb: 1/2 had large number of CD3+ cells (>than IgG1 group)
hIL-6: similar level of CD3+ cells as IgG1 group
Combo: 3/3 had large number of CD3+ cells (>than IgG1 group)

FoxP3 expression
IgG1: low # FoxP3+ cells scattered throughout tumor
mPD-1 mAb: 1/2 had high # at tumor periphery
hIL-6: 1/3 had high # at tumor periphery
combo: 2/3 mice in combo group may have slightly greater # within tumor than IgG1

CD31 expression: similar between all groups

### Example 6

### Recombinant Mouse IL-6 and Anti-Mouse PD-1 Monoclonal Antibody (mAb) in a Mouse Subcutaneous CT-26 Colon Carcinoma Model and Flow Cytometry Analysis of Tumors

### Materials and Methods

### Animals:

Female BALB/c mice (Harlan Laboratories, Livermore, Calif.) were acclimated for a minimum of three days prior to the start of the studies. Mice were housed 5 animals per cage, and the cages were placed in microisolator ventilated racks. Housing was at 18-26° C. and 50±20% relative humidity with at least twelve room air changes per hour. A 12 h light/dark cycle was maintained. Animals were provided with sanitized laboratory rodent diet and municipal water ad libitum.

### Preparation and Implantation of Tumor Cells

CT-26 cells were obtained from the BMS master cell bank and maintained in RPMI-1640 medium (Hyclone, Cat. No. SH30096.01) supplemented with 10% fetal bovine serum (FBS; Hyclone, Cat. No. SH30071.03). Approximately twice a week, cells contained in a single T175 flask were divided and expanded to four T175 flasks at a 1:5 dilution until sufficient number of cells were obtained for tumor implantation. The cells were harvested near 80% confluence, washed and resuspended in PBS (Hyclone, Cat. No. SH30256.01).

On Day 0, 1×10⁶ CT-26 cells were implanted into the mice using a 1 cc syringe (Becton Dickinson, Franklin Lakes, N.J.) and 27 gauge ⅝ inch needle. Tumors were then measured two times weekly in 3 dimensions with an electronic caliper (Mitutoyo, Aurora, Ill.) and recorded. Tumor volumes (mm³) were calculated using the formula: width×length×height×0.5.

### IL-6 and Antibody Treatments

Following tumor volume measurements on Day 8 post-tumor implantation, mice were staged according to tumor volume. Mice with a mean tumor volume of 35 mm³ were randomized into groups and treated as shown in Table 6.

The mouse (m)IgG1 isotype control antibody is an inert monoclonal antibody (mAb) obtained from a mouse hybridoma against glu-glu. It was prepared in PBS immediately prior to administration to provide doses of 7 mg/kg per mouse via intraperitoneal (IP) injection on Days 8, 11 and 14 as shown in Table 6.

The monoclonal antibody against mouse PD-1 (clone 4H2) was prepared in PBS immediately prior to administration to provide doses of 7 mg/kg per mouse via intraperitoneal (IP) injection on Days 8, 11 and 14 as shown in Table 6. This antibody was produced at Bristol-Myers Squibb and was a mouse IgG1 that had been engineered to not bind Fc receptors. It should be noted that the dose of mPD-1 mAb was reduced from 10 mg/kg to 7 mg/kg for this experiment in order to provide sub-optimal dosing that might better enable seeing combination efficacy in the group treated with mPD-1 mAb+mIL-6 over either treatment alone, i.e. to better ensure that the mPD-1 mAb was not overly efficacious so that combination efficacy would be possible to see if it existed.

Recombinant mouse IL-6 (mIL-6) (R&D Systems, carrier-free, Cat. No. 406-ML-025/CF) was prepared in 0.02% (w/v) mouse albumin (Sigma-Aldrich, Cat. No. A3139) immediately prior to administration via subcutaneous (SC) injection at doses of 3 micrograms/kg, 3 times per week, beginning on Day 8 as shown in Table 6.

TABLE 6
Experiment #6 Treatment Groups

| Treatment | N | Route | Dose | Treatment schedule |
|---|---|---|---|---|
| mIgG1 control | 15 | IP | 7 mg/kg | d. 8, 11, 14 |
| mPD-1 mAb | 15 | IP | 7 mg/kg | d. 8, 11, 14 |
| mIL-6 | 15 | SC | 3 μg/kg | d. 8, 11, 14, 16, |
| | | | | 19, 21, 23, 26 |
| mIL-6 | 15 | SC | 3 μg/kg | mIL-6: d. 8, 11, 14, |
| | | | mIL-6 | 16, 19, 21, 23, 26 |
| PD-1 mAb | | IP | 7 mg/kg | mPD-1 mAb: d. 8, |
| | | | mPD-1 mAb | 11, 14 |

m = mouse-specific

### Post-Treatment Monitoring

Animals were checked daily for postural, grooming, and respiratory changes, as well as lethargy. Animals were weighed two times weekly and euthanized if weight loss was ≥20%.

Mice were checked for the presence and size of tumors twice weekly until death or euthanasia. Tumors were measured in 3 dimensions with an electronic caliper (Mitutoyo, Aurora, Ill.) and recorded. Response to treatment compounds was measured as a function of tumor growth. If the tumor reached a volume of ≥1500 mm³ or appeared ulcerated, animals were euthanized.

### Flow Cytometry Analysis of Tumors

At Day 15, five mice per group that had tumor volumes representative of their group were sacrificed, tumors removed and processed to single cell suspensions using the Miltenyi GentleMacs mouse tumor enzymatic digestion system (Miltenyi Biotec Inc, San Diego, Calif.). Cells (2×106 cell/mouse) were filtered and resuspended in FACS buffer (PBS, 0.1% BSA, 2 mM EDTA) containing anti-CD16/CD32 Fc-block, stained with indicated antibodies, and collected on a Becton, Dickinson LSR Fortessa flow cytometer (Becton, Dickinson and Co, Franklin Lakes, N.J.). Data were analyzed with FlowJo analysis software (FlowJo LLC, Ashland, Oreg.). The remaining 10 mice/group were followed for anti-tumor activity.

### Statistical Analyses

Statistical analyses were performed using GraphPad Prism software (Version 5.01), with the statistical test that was utilized being noted in the figure legends. A p value <0.05 was considered statistically significant. Mean and median tumor volumes for groups were calculated while at least 70% of study animals remained alive.

### Results

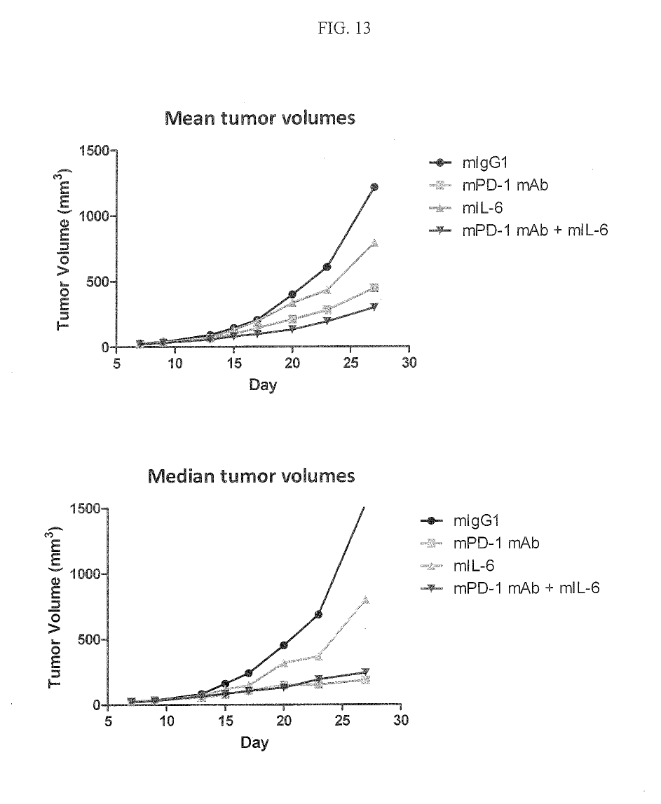

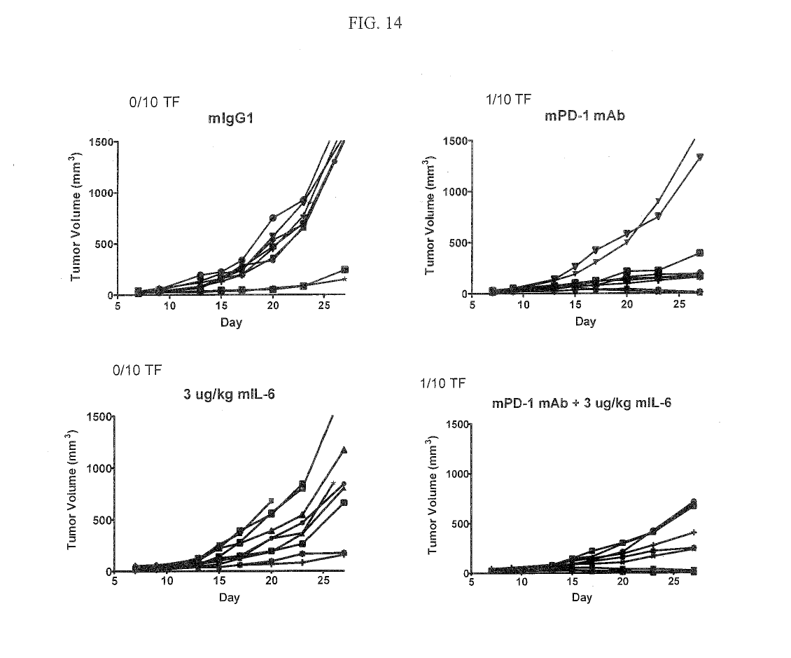

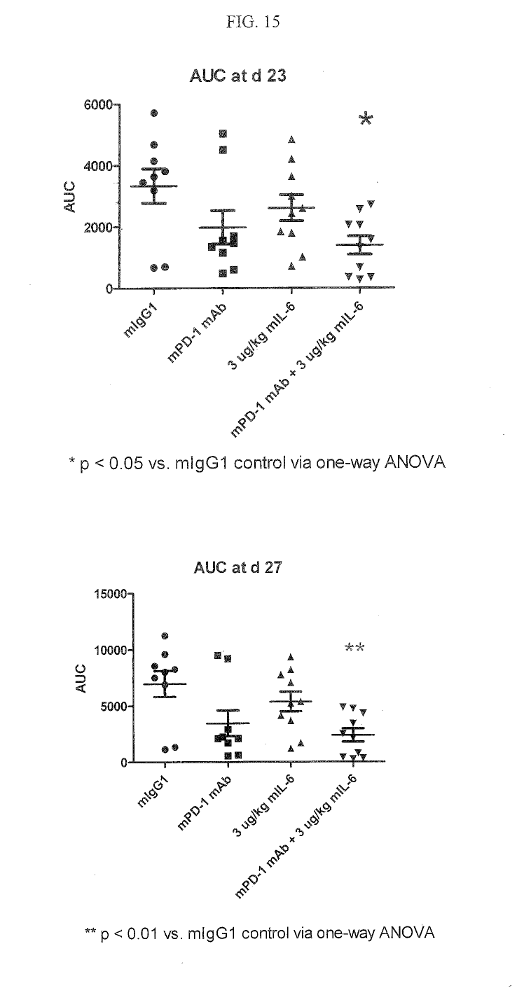

Mice treated with mPD-1 mAb or mIL-6 as single agents had some tumor growth delays as compared to the mIgG1 group as shown by the lower mean and median tumor volumes over time as compared to the group treated with mIgG1 control mAb (FIG. 13); the group treated with the combination of mPD-1 mAb and mIL-6 had mean and median tumor volumes that were lower than either of the two mono-therapy groups. At Day 27, there was 1/10 tumor-free mouse in the group treated with mPD-1 mAb as mono-therapy and no tumor-free mice in the mIgG1 control or mIL-6 treatment groups (FIG. 14). The group treated with the combination of mPD-1 mAb and mIL-6 also had 1 tumor-free mouse at Day 27 but as demonstrated by area-under-the-curve (AUC) analyses at Day 23 and Day 27, only the group of mice treated with the combination of mPD-1 mAb and mIL-6 had statistically significantly lower mean AUC values as compared to the mIgG1 control mAb group by one-way ANOVA (p<0.05 or <0.01 for Day 23 and Day 27, respectively); there were no other significant differences between groups by one-way ANOVA (FIG. 15).

In order to evaluate potential mechanisms for the enhanced antitumor efficacy observed with the combination of PD-1 mAb and IL-6, tumors from a representative subgroup of mice from each group (n=5 mice/group from the original 15/group) were evaluated by flow cytometry at Day 15 for levels of cell populations known to play a role in antitumor immunity. The group of mice treated with the combination of mPD-1 mAb and mIL-6 had a statistically significant lower percentage of Foxp3+ regulatory T cells (as a percentage of live CD45+ cells) in tumor infiltrating lymphocytes (TIL) as compared to the group treated with the mIgG1 control mAb (FIG. 15). Given that regulatory T (Treg) cells can provide immunosuppressive signals to tumors, treatments that can reduce Treg cells is well-accepted way to enhance antitumor activity. The combination treatment group also had a higher percentage of CD8+ T cells (as a percent of live CD45+ cells) and a higher percentage of AH1+ CD8+ T cells (as a percent of live CD45+ cells) as compared to the mIgG1 group though the differences were not statistically significant. CD8+ T cells provide cytotoxic antitumor activity and AH1 is a tumor antigen for CT-26 tumors. Thus, the observation in the group treated with the combination of mPD-1 mAb and mIL-6 of a lower percentage of Foxp3+ Treg cells, a higher percentage of CD8+ T cells, and a higher percentage of tumor antigen-specific CD8+ T cells in TIL provides potential mechanisms for the enhanced antitumor activity observed in this group.

The combination efficacy observed in this well-established pre-clinical tumor model further supports the idea that co-treatment with PD-1 mAb and recombinant IL-6 could be an effective method for the treatment of cancer, with an enhanced antitumor immunity providing potential mechanisms-of-action for this activity.

FIG. 13 shows the mean and median tumor volumes by group showing antitumor activity of mPD-1 mAb and mIL-6, alone or in combination, in the CT-26 tumor model.

FIG. 14 shows the individual mouse tumor volume data showing antitumor activity of mPD-1 mAb and mIL-6, alone or in combination, in the CT-26 tumor model. Each line represents an individual mouse. Red data points note that the mouse was euthanized due to tumor ulceration. TF=tumor-free. The mPD-1 mAb mono-therapy group and the group treated with the combination of mPD-1 mAb and mIL-6 had significantly lower (p<0.05) mean and median tumor volumes over time as compared to the mIgG1 group by two-way (repeated measures) ANOVA at Day 27, the latest day possible due to loss of mice from tumor burden or tumor ulceration.

FIG. 15 shows the area-under-the-curve (AUC) values for tumor volumes through Day 23 and Day 27 showing antitumor activity of mPD-1 mAb and mIL-6, alone or in combination, in the CT-26 tumor model. Day 27 was the latest day possible to analyze due to the loss of mice from tumor burden or tumor ulceration. Statistical analyses between all groups were conducted using one-way ANOVA, followed by Dunn's multiple comparison tests between groups: p<0.05 for mPD-1 mAb+mIL-6 combination vs. mIgG1 control (Day 23); p<0.01 for mPD-1 mAb+mIL-6 combination vs. mIgG1 control (Day 27).

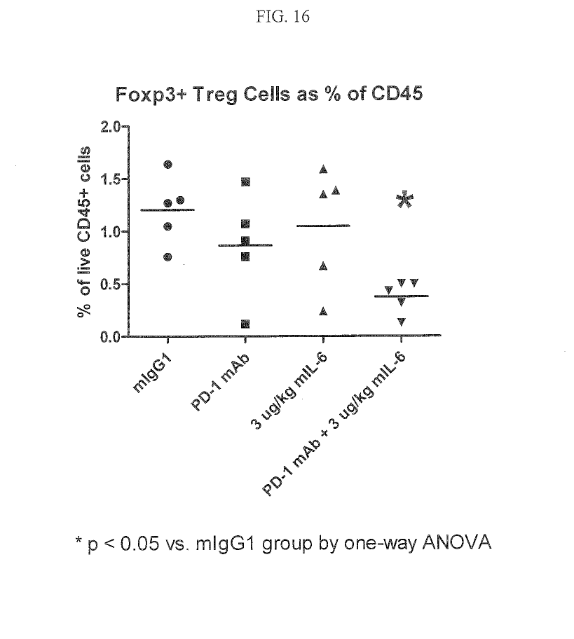

FIG. 16 shows the percentage of Foxp3+ regulatory T cells (Treg) as a proportion of live CD45+ cells in tumors (at Day 15) from mice treated with mIgG1, or mPD-1 mAb or mIL-6 alone and in combination in the CT-26 tumor model. Statistical analyses between all groups were conducted using one-way ANOVA, followed by Dunn's multiple comparison tests between groups: p<0.05 for mPD-1 mAb+mIL-6 combination vs. mIgG1 control.

### Example 7

### Two Dose Levels of Recombinant Mouse IL-6 and Anti-Mouse PD-1 Monoclonal Antibody (mAb) in a Mouse Subcutaneous CT-26 Colon Carcinoma Model

### Materials and Methods

### Animals:

Female BALB/c mice (Harlan Laboratories, Livermore, Calif.) were acclimated for a minimum of three days prior to the start of the studies. Mice were housed 5 animals per cage, and the cages were placed in microisolator ventilated racks. Housing was at 18-26° C. and 50±20% relative humidity with at least twelve room air changes per hour. A 12 h light/dark cycle was maintained. Animals were provided with sanitized laboratory rodent diet and municipal water ad libitum.

### Preparation and Implantation of Tumor Cells

CT-26 cells were obtained from the BMS master cell bank and maintained in RPMI-1640 medium (Hyclone, Cat. No. SH30096.01) supplemented with 10% fetal bovine serum (FBS; Hyclone, Cat. No. SH30071.03). Approximately twice a week, cells contained in a single T175 flask were divided and expanded to four T175 flasks at a 1:5 dilution until sufficient number of cells were obtained for tumor implantation. The cells were harvested near 80% confluence, washed and resuspended in PBS (Hyclone, Cat. No. SH30256.01).

On Day 0, 1×10⁶ CT-26 cells were implanted into the mice using a 1 cc syringe (Becton Dickinson, Franklin Lakes, N.J.) and 27 gauge ⅝ inch needle. Tumors were then measured two times weekly in 3 dimensions with an electronic caliper (Mitutoyo, Aurora, Ill.) and recorded. Tumor volumes (mm³) were calculated using the formula: width×length×height×0.5.

### IL-6 and Antibody Treatments

Following tumor volume measurements on Day 8 post-tumor implantation, mice were staged according to tumor volume. Mice with a mean tumor volume of 29 mm³ were randomized into groups and treated as shown in Table 7.

The mouse (m)IgG1 isotype control antibody is an inert monoclonal antibody (mAb) obtained from a mouse hybridoma against glu-glu. It was prepared in PBS immediately prior to administration to provide doses of 7 mg/kg per mouse via intraperitoneal (IP) injection on Days 8, 11, 14 and 21 as shown in Table 7.

The monoclonal antibody against mouse PD-1 (clone 4H2) was prepared in PBS immediately prior to administration to provide doses of 7 mg/kg per mouse via intraperitoneal (IP) injection on Days 8, 11, 14 and 21 as shown in Table 7. This antibody was produced at Bristol-Myers Squibb and was a mouse IgG1 that had been engineered to not bind Fc receptors. It should be noted that the dose of mPD-1 mAb was again 7 mg/kg for this experiment, a dose used to provide sub-optimal dosing that might better enable seeing combination efficacy in the groups treated with mPD-1 mAb+mIL-6 as compared to the treatments as mono-therapy, i.e. to better ensure that the mPD-1 mAb was not overly efficacious so that combination efficacy would be possible to see if it existed. An additional dose of mPD-1 mAb was administered on Day 21 to extend its exposure in the mice and in order to mimic what might be done clinically.

Recombinant mouse IL-6 (mIL-6) (R&D Systems, carrier-free, Cat. No. 406-ML-025/CF) was prepared in 0.02% (w/v) mouse albumin (Sigma-Aldrich, Cat. No. A3139) immediately prior to administration via subcutaneous (SC) injection at doses of 3 or 30 micrograms/kg, 3 times per week, beginning on Day 8 as shown in Table 7.

TABLE 7
Experiment #7 Treatment Groups

| Treatment | N | Route | Dose | Treatment schedule |
|---|---|---|---|---|
| mIgG1 control | 15 | IP | 7 mg/kg | d. 8, 11, 14, 21 |
| mPD-1 mAb | 15 | IP | 7 mg/kg | d. 8, 11, 14, 21 |
| mIL-6 | 15 | SC | 3 μg/kg | d. 8, 11, 14, 16, 19, |
| | | | | 21, 23, 26 |
| mIL-6 | 15 | SC | 3 μg/kg | mIL-6: d. 8, 11, 14, |
| | | | mIL-6 | 16, 19, 21, 23, 26 |
| PD-1 mAb | | IP | 7 mg/kg | mPD-1 mAb: d. 8, 11, |
| | | | mPD-1 mAb | 14, 21 |
| mIL-6 | 15 | SC | 30 μg/kg | d. 8, 11, 14, 16, 19, |
| | | | | 21, 23, 26 |
| mIL-6 | 15 | SC | 30 μg/kg | mIL-6: d. 8, 11, 14, |
| | | | mIL-6 | 16, 19, 21, 23, 26 |
| PD-1 mAb | | IP | 7 mg/kg | mPD-1 mAb: d. 8, 11, |
| | | | mPD-1 mAb | 14, 21 |

m = mouse-specific

### Post-Treatment Monitoring

Animals were checked daily for postural, grooming, and respiratory changes, as well as lethargy. Animals were weighed two times weekly and euthanized if weight loss was ≥20%.

Mice were checked for the presence and size of tumors twice weekly until death or euthanasia. Tumors were measured in 3 dimensions with an electronic caliper (Mitutoyo, Aurora, Ill.) and recorded. Response to treatment compounds was measured as a function of tumor growth. If the tumor reached a volume of ≥1500 mm³ or appeared ulcerated, animals were euthanized.

### Analysis of Interferon Gamma (IFNγ) Concentrations in Tumors

At Day 15, five mice per group that had tumor volumes representative of their group were sacrificed, tumors removed and processed to single cell suspensions using the Miltenyi GentleMacs mouse tumor enzymatic digestion system (Miltenyi Biotec Inc, San Diego, Calif.). The cell suspension were centrifuged for 5 minutes, 1500×g after which the supernatants were collected and frozen at

−80° C. until analyzed for concentrations of mouse IFNγ using Luminex-based methodology. Concentrations were corrected for by the weight of the tumor. The remaining 10 mice/group were followed for anti-tumor activity.

### Statistical Analyses

Statistical analyses were performed using GraphPad Prism software (Version 5.01), with the statistical test that was utilized being noted in the figure legends. A p value <0.05 was considered statistically significant. Mean and median tumor volumes for groups were calculated while at least 70% of study animals remained alive.

### Results

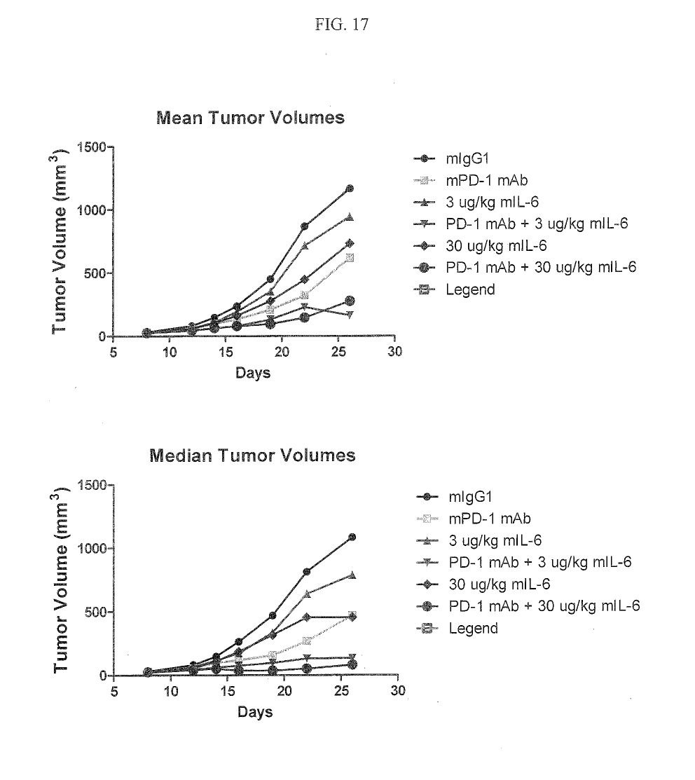

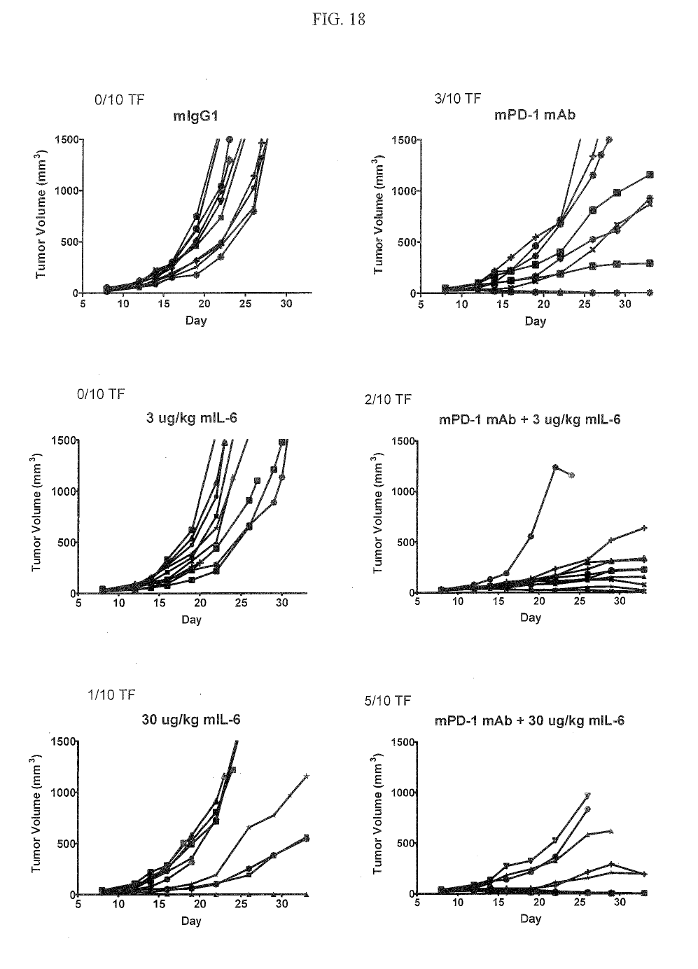

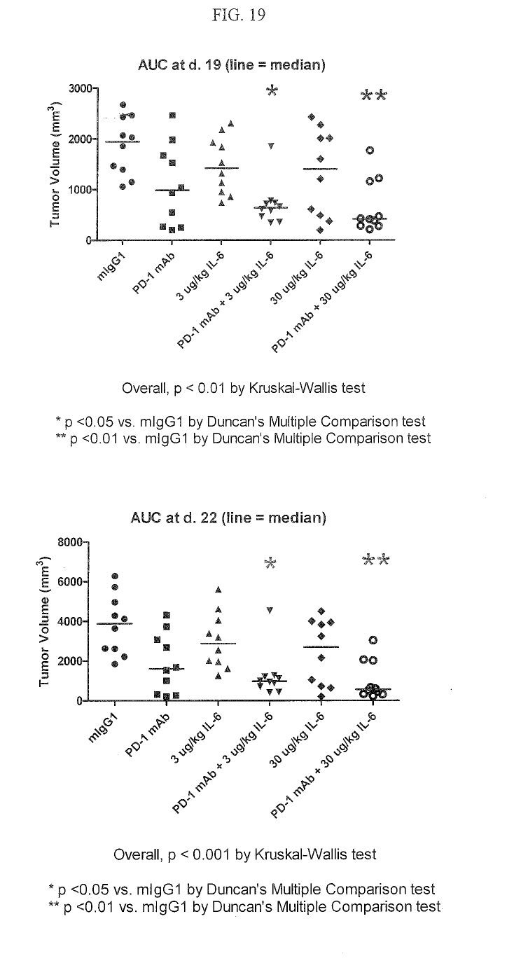

Mice treated with mPD-1 mAb or mIL-6 as single agents had some tumor growth delays as compared to the mIgG1 group as shown by the lower mean and median tumor volumes over time as compared to the group treated with mIgG1 control mAb (FIG. 17); the group treated with 30 μg/kg mIL-6 had lower mean and median tumor volumes than the group treated with 3 μg/kg mIL-6. The groups treated with the combination of mPD-1 mAb and either 3 or 30 μg/kg mIL-6 had mean and median tumor volumes that were lower than any of the mono-therapy groups (FIG. 17). At Day 33, there were 3/10 tumor-free mouse in the group treated with mPD-1 mAb as mono-therapy and no tumor-free mice in the mIgG1 control or 3 μg/kg mIL-6 mono-therapy groups; there was 1/10 tumor-free mice in the 30 μg/kg group (FIG. 18). The group treated with the combination of mPD-1 mAb and 3 μg/kg mIL-6 had 2 tumor-free mice at Day 33, as well as a mouse with a very small tumor (<15 mm³) that would likely become tumor-free. There was an IL-6 dose-dependent effect on tumor rejection in the combination groups, with the 30 μg/kg combination group having 5/10 tumor-free mice at Day 33 (FIG. 18). Both groups treated with the combination of mPD-1 mAb and mIL-6 (i.e. 3 and 30 μg) had statistically significantly lower mean AUC values for tumor volumes as compared to the mIgG1 control mAb group by one-way ANOVA (p<0.05 for the 3 μg/kg mIL-6 combination group and p<0.01 for the combination group with 30 μg/kg mIL-6) at both Day 19 and Day 22 (the latest day possible due to loss of mice in some groups from tumor burden or tumor ulceration) (FIG. 19).

In order to evaluate additional potential mechanisms for the enhanced antitumor efficacy observed with the combination of PD-1 mAb and IL-6, levels of IFNγ in tumor supernatants were evaluated from a representative subgroup of mice from each group (n=5 mice/group from the original 15/group) at Day 15. This cytokine is well-known to play a role promoting antitumor immunity and is a mechanism responsible for the antitumor activity of anti-PD-1 therapy. Each of the groups treated with mPD-1 mAb (whether alone or in combination) had increases in tumor IFNγ levels above those of mIgG1 control-treated mice, with the addition of mIL-6 maintaining the enhancement. The groups treated with the mIL-6 as monotherapy had levels of IFNγ that were slightly higher than the mIgG1-treated group. The enhanced IFNγ levels and associated antitumor immunity that it affords may be an additional mechanism provided to IL-6 for its ability to further reduce tumor growth in combination with PD-1 blockade.

The combination efficacy observed in this well-established pre-clinical tumor model further supports the idea that co-treatment with PD-1 mAb and recombinant IL-6 could be an effective method for the treatment of cancer, with an enhanced antitumor immunity providing potential mechanisms-of-action for this activity.

FIG. 17 shows the mean and median tumor volumes by group showing antitumor activity of mPD-1 mAb and mIL-6, alone or in combination, in the CT-26 tumor model. The mPD-1 mAb mono-therapy group and the group treated with 30 μg/kg mIL-6 as mono-therapy had significantly lower (p<0.05) mean and median tumor volumes over time as compared to the mIgG1 group by two-way (repeated measures) ANOVA at Day 26 (the latest day possible due to loss of mice from tumor burden or tumor ulceration); the groups treated with the combination of mPD-1 mAb and either 3 or 30 μg/kg mIL-6 had lower mean and median tumor volumes that were significantly different at p<0.01 vs. mIgG1 control group by the same analysis.

FIG. 18 shows the individual mouse tumor volume data showing antitumor activity of mPD-1 mAb and mIL-6, alone or in combination, in the CT-26 tumor model. Each line represents an individual mouse. Red data points note that the mouse was euthanized due to tumor ulceration.

FIG. 19 shows the area-under-the-curve (AUC) values for tumor volumes through Day 19 and Day 22 showing antitumor activity of mPD-1 mAb and mIL-6, alone or in combination, in the CT-26 tumor model. Day 22 was the latest day possible to analyze due to the loss of mice from tumor burden or tumor ulceration. Statistical analyses between all groups were conducted using one-way ANOVA, followed by Dunn's multiple comparison tests between groups: p<0.05 for mPD-1 mAb+3 μg/kg mIL-6 combination vs. mIgG1 control (Day 19 and Day 22); p<0.01 for mPD-1 mAb+30 μg/kg mIL-6 combination vs. mIgG1 control (Day 19 and Day 22).

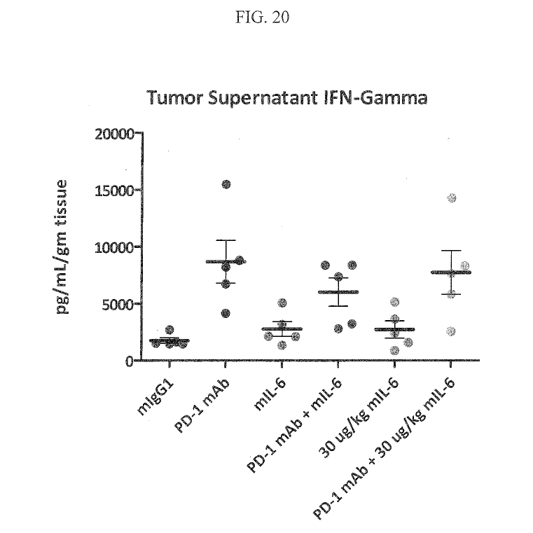

FIG. 20 shows the concentration of IFNγ in tumor supernatants at Day 15, and expressed as pg/mL per gram tumor weight. The mean differences between groups were not statistically different.

Taken together, the results from these studies demonstrate that co-treatment with PD-1 blockade and recombinant IL-6 is more efficacious than either treatment alone in mouse models of cancer, and support the idea that PD-1 blockade in combination with recombinant human IL-6 may be an effective treatment of cancer in humans.

### LITERATURE CITED

Andriambeloson E, Baillet C, Vitte P A, Garotta G, Dreano M, Callizot N. Interleukin-6 attenuates the development of experimental diabetes-related neuropathy. Neuropathology (2006) 26:32-42.
Armstrong C A, et al. Melanoma-derived interleukin 6 inhibits in vivo melanoma growth. J Invest Dermatol (1994) 102:278-284.
Bhanumathy K K, et al. Transgene IL-6 enhances DC-stimulated CTL responses by counteracting CD4+25+Foxp3+ regulatory T cell suppression via IL-6-induced Foxp3 downregulation. Int J Mol Sci (2014) 15:5508-5521.
Burnet F M. Cancer—A Biological Approach: I. The Processes Of Control. II. The Significance of Somatic Mutation. Brit Med Jour (1957) 1: 779-786.
Callizot N, et al. Interleukin-6 protects against paclitaxel, cisplatin and vincristine-induced neuropathies without impairing chemotherapeutic activity. Cancer Chemother Pharmacol (2008) 62:995-1007.
Cao X, et al. Induction of antitumor immunity and treatment of pre-established tumor by interleukin-6-gene-transfected melanoma cells combined with low-dose interleukin-2. J Cancer Res Clin Oncol (1995) 121:721-728.
Dunn G, et al. Cancer immunoediting: from immunosurveillance to tumor escape. Nature Immunology (2002) 3: 991-998.
Dunn G P, et al. The Three Es of Cancer Immunoediting. Ann Rev Immunol (2004) 22:329-360.
Dunn G P, et al. The immunobiology of cancer immunosurveillance and immunoediting. Immunity. (2004a) 21:137-148.
Eisenthal A, et al. Antitumor effects of recombinant interleukin-6 expressed in eukaryotic cells. Cancer Immunol Immunother (1993) 36:101-107.
Gajewsky T F, et al. Costimulation with B7.1, IL-6, and IL-12 is sufficient for primary generation of murine antitumor cytolytic T lymphocytes in vitro. J Immunol (1995) 154: 5637-5648.
Gilbert, L A and Hemann, M T. Context-specific roles for paracrine IL-6 in lymphomagenesis. Genes Dev (2012) 26, 1758-1768.
Givon T, Slavin S, Haran-Ghera N, et al. Antitumor effects of human recombinant interleukin-6 on acute myeloid leukemia in mice and in cell cultures. Blood (1992) 79: 2392-2398.
Grivennikov S I, Greten F R, Karin M. Immunity, inflammation, and cancer. Cell (2010) 140:883-899.
Hirano T, Ishihara K, Hibi M. Roles of STAT3 in mediating the cell growth, differentiation and survival signals relayed through the IL-6 family of cytokinereceptors. Oncogene (2000) 19:2548-2556.
Kim R, et al. Cancer immunoediting from immune surveillance to immune escape. J Immunol (2007) 121: 1-14.
Kishimoto T. The biology of interleukin-6. Blood (1989) 74: 1-10.
Kitahara M, Kishimoto S, Hirano T, Kishimoto T, Okada M: The in vivo antitumor effect of human recombinant interleukin-6. Jpn J Cancer Res (1990) 81:1032.
Korman A, et al. Checkpoint blockade in cancer immunotherapy. Adv Immunol (2006) 90:297-339.
Le J and Vilcek J. Biology of disease. Interleukin 6: a multifunctional cytokine regulating immune reactions and the acute phase protein response. Lab Invest (1989) 61: 588-602.
Lin G, et al. Interleukin-6 inhibits regulatory T cells and improves the proliferation and cytotoxic activity of cytokine-induced killer cells. J Immunother (2012) 35:337-343.
Marcus S G, et al. The use of interleukin-6 to generate tumor-infiltrating lymphocytes with enhanced in vivo antitumor activity. J Immunother Emphasis Tumor Immunol (1994)15:105-112.
Mulé J J, et al. Antitumor activity of recombinant interleukin 6 in mice. J Exp Med (1990) 171: 629-636.
Mullen C A, et al. Fibrosarcoma cells transduced with the IL-6 exhibit reduced tumorigenicity, increased immunogenicity, and decreased metastatic potential. Cancer Res (1992) 52: 6020-6024.
Naugler W E, Karin M. The wolf in sheep's clothing: the role of interleukin-6 in immunity, inflammation and cancer. Trends Mol Med (2008) 14:109-119.
Nordon R P, et al. Purification and NH2-terminal sequence of a plasmacytoma growth factor derived from the murine macrophage cell line P388D1. J Immunol (1987) 139:813-817.
O'Sullivan T, et al. Cancer immunoediting by the innate immune system in the absence of adaptive immunity. JEM (2012) 209:1869-1882.
Pardoll D M. The blockade of immune checkpoints in cancer immunotherapy. Nature Reviews Cancer (2012) 12: 252-264.
Pedersen L, Idom M, Olofsson G H, Lauenborg B, Nookaew I, Hansen R H, Johannesen H H, Becker J C, Pedersen K S, Dethlefsen C, Nielsen J, Gehl J, Pedersen B K, Thor Straten P, Hojman P. Voluntary running suppresses tumor growth through epinephrine- and IL-6-dependent NK cell mobilization and redistribution. Cell Metab. 2016 March 8; 23(3):554-562.
Progador A, et al. Interleukin 6 gene transfection into Lewis lung carcinoma tumor cells suppresses the malignant phenotype and confers immunophenotypic competence against parental metastatic cells. Cancer Res (1992) 52: 3679-3686.
Qu S, et al. Preliminary study on the cooperation of IL-6 and mB7-1 in the induction of effective antitumor immunity in vitro. J Tongji Med Univ (1999) 19:86-89.
Riella L V, et al. Role of the PD-1 pathway in the immune response. Am J Transplant (2012) 12:2575-2587.
Rose-John S. IL-6 trans-signaling via the soluble IL-6 receptor: importance for the pro-inflammatory activities of IL-6. Int J Biol Sci (2012) 8:1237-1247.
Shankaran V H, et al. IFNgamma and lymphocytes prevent primary tumour development and shape tumour immunogenicity. Nature (2001) 410:1107-1111.
Silver J S, Hunter C A. gp130 at the nexus of inflammation, autoimmunity, and cancer. J Leukoc Biol (2010) 88:1145-1156.
Sun W H, Kreisle R A, Phillips A W, Ershler W B. In vivo and in vitro characteristics of interleukin 6-transfected tumor B16 melanoma cells. Cancer Res (1992) 52: 5412-5415.
Topalian S L, et al. Targeting the PD-1/B7-H1(PD-L1) pathway to activate antitumor immunity. Curr Opin Immunol (2012) 24:207-212.
Veldhuis G J, et al. Potential Use of Recombinant Human Interleukin-6 in Clinical Oncology. Leukemia and Lymphoma (1996) 20: 373-379.

What is claimed is:

1. A method for treating a subject afflicted with a cancer comprising administering to the subject a therapeutically effective amount of:
(a) an anti-cancer agent which is an antibody or an antigen-binding portion thereof that binds specifically to a Programmed Death-1 (PD-1) receptor and inhibits PD-1 activity which is administered by infusion for less than 60 minutes; and
(b) soluble IL-6.

2. The method of claim 1, wherein the cancer is selected from the group consisting of lung cancer, renal cancer and metastatic melanoma.

3. The method of claim 1, wherein the anti-PD-1 antibody or antigen-binding portion thereof cross-competes with nivolumab for binding to human PD-1.

4. The method of claim 3, wherein the anti-PD-1 antibody or antigen-binding portion thereof is a chimeric, humanized or human monoclonal antibody or a portion thereof.

5. The method of claim 4, wherein the anti-PD-1 antibody or antigen-binding portion thereof comprises a heavy chain constant region which is of a human IgG1 or IgG4 isotype.

6. The method of claim 1, wherein the anti-PD-1 antibody is nivolumab.

7. The method of claim 1, wherein the anti-PD-1 antibody is pembrolizumab.

8. The method of claim 5, wherein the anti-PD-1 antibody or antigen-binding portion thereof is administered at a dose ranging from 0.1 to 10.0 mg/kg body weight once every 2 or 3 weeks.

9. The method of claim 8, wherein the anti-PD-1 antibody or antigen-binding portion thereof is administered at a dose of 1 or 3 mg/kg body weight once every 2 weeks or once every 3 weeks.

10. The method of claim 9, wherein the anti-PD-1 antibody or antigen-binding portion thereof is administered at a dose of 1 mg/kg body weight once every 3 weeks.

11. The method of claim 9, wherein the anti-PD-1 antibody or antigen-binding portion thereof is administered at a dose of 3 mg/kg body weight once every 2 weeks.

12. The method of claim 11, wherein the anti-PD-1 antibody or antigen-binding portion is administered for as long as clinical benefit is observed or until unmanageable toxicity or disease progression occurs.

13. The method of claim 1, wherein the IL-6 is human IL-6.

14. The method of claim 13, wherein the IL-6 is produced recombinantly.

15. The method of claim 14, wherein the IL-6 may be glycosylated or non-glycosylated.

16. The method of claim 15, wherein the dose of IL-6 is in the range of 0.06 to 3 μg/kg body weight, preferably in the range of 0.1 to 2 μg/kg body weight. The dose of IL-6 is in the range of 4 to 210 μg, preferably in the range of 7 to 140 μg.


SEQUENCE LISTING (2 sequences)

SEQ ID NO: 1 - DNA, 2555 bp, Homo sapiens
cccgctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttc

SEQ ID NO: 2 - protein, 252 aa, Homo sapiens
MNSFSTSKCRKSLALELPAAVEPCVREGCVAQGGLAGGQQQRQAPSCAVSSPLRSLPSGTGAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKVGVSSFPQLGVGEDRLKDSVLDNSGMQCHFQKRRLHVNKRV